(19) United States
(12) Patent Application Publication
(10) Pub. No.: US 2026/0078333 A1
(43) Pub. Date: Mar. 19, 2026

(54) METHOD OF ISOLATING ANIMAL GONADAL MESENCHYMAL STEM CELLS

(71) Applicants: Amanda Baracho Trindade Hill, Raleigh, NC (US); Jonathan Edwin Baracho Trindade Hill, raleigh, NC (US)

(72) Inventors: Amanda Baracho Trindade Hill, Raleigh, NC (US); Jonathan Edwin Baracho Trindade Hill, raleigh, NC (US)

(21) Appl. No.: 19/312,454

(22) Filed: Aug. 28, 2025

Related U.S. Application Data
(60) Provisional application No. 63/695,952, filed on Sep. 18, 2024.

(51) Int. Cl. C12M 1/00; A61K 35/28; C12N 5/0775

(52) U.S. Cl. CPC C12M 47/04; A61K 35/28; C12N 5/0668; C12N 2510/00

(57) ABSTRACT

A method for the isolation of cells in animal gonadal tissue. These stem cells can be used in a variety of treatments where these specific stem cells work.

### RELATED U.S. APPLICATION DATA

This application claims benefit of provisional U.S. application No. 63/695,952 filed Sep. 18, 2024.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a method of isolating animal gonadal mesenchymal cells for use in treating a mammal.

### Description of Related Art

The Mesenchymal stem cells (MSCs) also known as mesenchymal stromal cells or medicinal signaling cells, are multipotent stromal cells that can differentiate into a variety of cell types, including osteoblasts (bone cells), chondrocytes (cartilage cells), myocytes (muscle cells), adipocytes (fat cells which give rise to marrow adipose tissue), hepatocyte and neurogenic lineages. Each source of mesenchymal stem cells produces different results in the treatment of a medical condition,

MSCs are found throughout the human body. However, for use in treating a medical condition there are a limited number of locations of MSCs used. Bone marrow was the original source of MSCs, and is still the most frequently utilized source. These bone marrow stem cells do not contribute to the formation of blood cells, and so do not express the hematopoietic stem cell marker CD34. They are sometimes referred to as bone marrow stromal stem cells. The youngest and most primitive MSCs may be obtained from umbilical cord tissue, namely Wharton's jelly and the umbilical cord blood. However, MSCs are found in much higher concentration in the Wharton's jelly compared to cord blood, which is a rich source of hematopoietic stem cells. The umbilical cord is available after a birth. It is normally discarded, and poses no risk for collection. These MSCs may prove to be a useful source of MSCs for clinical applications, due to their primitive properties and fast growth rate. Adipose-tissue-derived MSCs (AdMSCs), in addition to being easier and safer to isolate than bone-marrow-derived MSCs, can be obtained in larger quantities. The developing tooth bud of the mandibular third molar is a rich source of MSCs. While they are described as multipotent, it is possible that they are pluripotent. They eventually form enamel, dentin, blood vessels, dental pulp, and nervous tissues. These stem cells are capable of differentiating into chondrocytes, cardiomyocytes, melanocytes, and hepatocyte-like cells in vitro. Stem cells are also present in amniotic fluid.

### BRIEF SUMMARY OF THE INVENTION

The present invention relates to the discovery that gonadal mesenchymal stem cells (GMSC) can be isolated and poses different properties than previously used mesenchymal stem cells. They therefore GMSC's can be utilized to treat a number of medical conditions. Mesenchymal animal ovary stem cells are better for cell differentiation and showed cell markers when compared with adipose cells derived MSC.

Methods for using GMSC for treating patients with: Musculoskeletal Conditions, including but not limited to soft tissue injury, cardiomyopathies, muscular dystrophy, bone marrow disorders, Osteoarthritis; Neurological and Spinal Conditions including but not limited to spinal cord injury, intervertebral disc disease; Diseases of Internal Organs including but not limited to lung diseases, nephropathies, liver diseases, endometritis and endometriosis; Idiopathic and Autoimmune Conditions, including but not limited to inflammatory bowel disease, atopic dermatitis, diabetes, gingivostomatitis, lupus; Ophthalmic Conditions, including but not limited to keratitis, corneal ulcer, macular degeneration; Other Conditions, including Urinary Incontinence and fecal incontinence and the like.

According to certain aspect of some embodiments of the present invention, there is provided a pharmaceutical composition provided comprising the GMSC (genetically modified or not) and/or their derived vesicles, exosomes, and conditioned media as a biologically active fraction thereof, and a pharmaceutically acceptable excipient, diluent or carrier. In yet other embodiments, the bioactive agent is administered by a route comprising systemic administration or local administration at the site of tissue regeneration.

The invention provides treatment and/or alleviation of the conditions, the GMSC are autologous, allogenic or xenogeneic to the patient's species. In one aspect, the treatment is a xenotransplantation with minimal rejection or inflammation from the GMSC treatment with GMSC.

In another aspect, the invention provides for a bank of veterinary species GMSC for use in treatment of a medical condition wherein the GMSC are xenogeneic to the recipient of the GMSC and wherein the medical condition is one or more than one condition selected from the group consisting of but not limited to excluding others urinary incontinence, osteoarthritis, degenerative myelopathy, diabetes, tissue regeneration, wound healing, scarring, soft tissue defect, fecal incontinence, dilated cardiomyopathy, hip dysplasia, avascular necrosis of the femoral head, ligament injury, tendon injury, spinal cord injury, atherosclerosis-related infarctions, arthritis, muscular dystrophy, distemper virus, cognitive dysfunction, dementia, alzheimer, granulomatous meningoencephalitis, intervertebral disc disease, spinal cord injury, bone marrow aplasia, non-regenerative anemia, atopic dermatitis, nephropathies and acute and chronic kidney disease, hepatitis, inflammatory bowel disease, endometritis, inflammatory heart disease, keratitis, gingivostomatitis, bronchitis, asthma, pneumonia, fibrosis, lupus, myopathies, tendinopathy, and arthritis.

In another aspect, the invention provides for a bank of veterinary species GMSC for use in treatment of a medical condition wherein the GMSC are allogeneic to the recipient of the GMSC and wherein the medical condition is one or more than one condition selected from the group consisting of but not limited to excluding others urinary incontinence, osteoarthritis, degenerative myelopathy, diabetes, tissue regeneration, wound healing, scarring, soft tissue defect, fecal incontinence, dilated cardiomyopathy, hip dysplasia, avascular necrosis of the femoral head, ligament injury, tendon injury, spinal cord injury, atherosclerosis-related infarctions, arthritis, muscular dystrophy, distemper virus, cognitive dysfunction, dementia, alzheimer, granulomatous meningoencephalitis, intervertebral disc disease, spinal cord injury, bone marrow aplasia, non-regenerative anemia, atopic dermatitis, nephropathies and acute and chronic kidney disease, hepatitis, inflammatory bowel disease, endometritis, inflammatory heart disease, keratitis, gingivostomatitis, bronchitis, asthma, pneumonia, fibrosis, lupus, myopathies, tendinopathy, and arthritis.

Optionally, any of the above GMSC banks of GMSC above can be administered using non excluding examples with hydrogel, hyaluronic acid, nanoparticles and other substances.

Additional embodiments comprise induction of GMSC biofactors to modulate their therapeutic benefit, for example to increase their ability to suppress or enhance an immune response, one or to more than one (i.e., to at least one) of the grammatical object of the article.

Accordingly, in one embodiment, there is a method of isolating animal gonadal mesenchymal stem cells comprising:
a) isolating animal gonadal tissue;
b) selecting mesenchymal stem cells in the animal gonadal tissue based on their rapid adherence to culture plastic; and
c) isolating the selected mesenchymal stem cells.

In another embodiment, there is a composition for treating a physical condition in an animal or human comprising animal gonadal mesenchymal stem cells isolated from animal gonadal tissue.

### DETAILED DESCRIPTION OF THE INVENTION

While this invention is susceptible to embodiment in many different forms, there is shown in the drawings, if there are any, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publication may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, “Mesenchymal stem cells” refers to fibroblast-like morphology cells, that express a combination of cell surface proteins that can include, but are not limited to, stem cell markers, including CD 90, the tetraspan protein CD9, CALLA (CD10), aminopeptidase N (CD13), integrin 1 (CD29), hyaluronate receptor (CD44), integrin alpha. 4 and 5 (CD49d, CD49e), ICAM-1 (CD54), decay accelerating factor (CD55), complement protectin (CD59), endoglin (CD105), VCAM-1 (CD106), Muc-1.8 (CD146), and ALCAM (CD166) In one aspect, GMSC are generally positive for CD90, CD44, CD73, CD105 and generally negative for CD34 and CD45 a. According to the international society of cellular therapy the isolated MSC should be positive for at least 2 MSC markers, negative for at least 2 hematopoietic markers, be able to self-renewal and differentiate into adipogenic, chondrogenic and osteogenic lineages.

As used herein, “Gonadal derived mesenchymal stem cells” refers to ovarian derived mesenchymal stem cells—(OMSC), and “testis derived mesenchymal stem cells—TMSC” which are used interchangeably herein and refer to stromal cells that originate from ovarian or testicular tissue and are capable of self-renewal and to differentiate in other cell types in non-limiting examples osteogenic, chodrongenic and adipogenic differentiation. GMSC can be derived from veterinary patients for their own treatment (i.e., autologous tissue) or from a clone of the patient, or from a different species (i.e., xenogeneic) or from the same species but not from the individual to be treated (i.e., allogeneic).

As used herein, the term “derived from” or “isolated from” shall be taken to indicate the tissue that the cell was isolated from and that came from, for example, “their derived conditioned media”, meaning that the conditioned media came from the GMSC.

As used herein, the term “tissue” refers to an aggregation of similarly specialized cells united in the performance of a particular function. Tissue is intended to encompass all types of biological tissue including both hard and soft tissue. Soft tissue refers to tissues that connect, support, or surround other structures and organs of the body. Soft tissue includes muscles, tendons (bands of fiber that connect muscles to bones), fibrous tissues, fat, blood vessels, nerves, and synovial tissues (tissues around joints). Hard tissue includes connective tissue (e.g., hard forms such as osseous tissue or bone) as well as other muscular or skeletal tissue.

As used herein “castration or isolating animal gonadal tissue” refers to the removal of gonadal tissue and accessory tissues. In males, this includes the removal of the testis in an operation commonly referred to as an orchiectomy (neuter surgery). In females, this includes the removal of the ovaries, and may also include the removal of the fallopian tubes or parts of all of the uterus, in an operation commonly referred to as an ovariectomy, or ovariohysterectomy (spay surgery).

As used herein, the term “preparation” or “purified preparation” or “isolation of GMSC” refers to a preparation of one or more cells that has been manipulated to provide a preparation of cells that is substantially free of additional components. In some aspects, the cell preparation is at least about 60%, by weight or number, free from other components that are present when the cell is produced. In various aspects, the cell preparation is at least about 55%, at least about 60%, at least about 65% at least about 70%, at least about 75%, at least about 80%, at least about 85%, at least about 90%, 91%, 92%, 93%, 94%, 95%, 96%, 97%, 98% or at least about 99%, by weight or number, pure. A purified cell preparation can be obtained, for example in non-limiting examples by from a natural source, fluorescence activated cell-sorting (FACS), or other techniques known to the skilled artisan, including based on cell adherence time, as provided for by this invention.

Purity can be assayed by any appropriate method, such as by FACS or by visual examination. “Purity”, as used to describe the purity of stem cells, does not refer to the presence of only stem cells in the composition but rather indicates that the stem cells have been manipulated such that they have been removed from their natural tissue environment, and indicates their relationship to the other cells present in the resulting population.

As used herein, the term “subpopulation” in the context of GMSC refers to a population of the primary isolated GMSC.

As used herein “non-human”, “veterinary species” and “animal” refers to farm animals, sport animals, pets, non-human primates, mice, and rats. Farm animals can include, but are not limited to, pigs, cows, horses, goats, and sheep. Pets include dogs, cats, rabbits, ferrets, and the like. Other animals within the scope of this definition include monkey, baboon, chimpanzee, orangutan, panda, tiger, lion, bear, cheetah, llama, and the like. Veterinary species refers to all animals that exist and can include all monkey, baboon, chimpanzee, orangutan, panda, tiger, lion, bear, cheetah, llama, turtles, birds, snakes, mice, rats, pigs, cows, horses, goats, sheep, dogs, cats, rabbits, ferrets and the like.

As used herein “patient”, “subject,” or “host” to be treated by the subject method cells can mean, for example, a human or non-human animal, such as a mammal, a fish, a bird, a reptile, or an amphibian, that is ill, at risk of illness, discomfort and need to improve health.

As used herein, “in need thereof” refers to patients (animal or human) who have a condition or disease or are “at risk” for the condition or disease. That physical condition can be from a number of conditions including autoimmune diseases, CNS diseases, allergic diseases, inflammatory diseases and the like.

As used herein, an “at risk” patient is an animal or human who is at risk of development of a physical condition. A patient “at risk” may or may not have a detectable condition, and may or may not have displayed detectable condition prior to the treatment methods described herein. “At risk” denotes that a patient has one or more so-called risk factors, which are measurable parameters that correlate with development of a disease or condition and are known in the art. A patient having one or more of these risk factors has a higher probability of developing the disease or condition than a subject without these risk factor(s). These risk factors include age, sex, diet, history of previous disease, presence of precursor disease, genetic (i.e., hereditary) considerations, breeding protocols and considerations, and environmental exposure.

The term “treatment” or “treating” refers to any therapeutic intervention method in a patient, for obtaining beneficial or desired results, including: (i) prevention, that is, causing the clinical symptoms not to develop, in a non-limiting example, preventing infection or inflammation from occurring and/or developing to a harmful state; (il) inhibition, that is, arresting the development of clinical symptoms, in a non-limiting example, stopping an ongoing infection so that the infection is eliminated completely or to the degree that it is no longer harmful; and/or (iii) relief, that is, causing the regression of clinical symptoms, e.g., causing a relief of fever and/or inflammation caused by or associated with a microbial infection.

For purposes of this invention, “beneficial” or “desired results” include the stabilization or improvement of the health-related quality of life of the patient, alleviation of symptoms associated with a condition diminishment of the extent of the symptoms associated with a condition, prevention of a worsening of the symptoms associated with a condition, or delaying the development of a disease or condition and the like. In some aspects, treatment with one or more cells disclosed herein is accompanied by no or fewer side effects than are associated with currently available therapies.

As used herein, “delaying development of a disease or condition” means to defer, hinder, slow, retard, stabilize and/or postpone development of the disease or condition. This delay can be of varying lengths of time, depending on the history of the disease and/or Subject being treated. As is evident to one skilled in the art, a sufficient or significant delay can, in effect, encompass prevention, in that the Subject does not develop the disease or condition. For example, the method may reduce the probability of disease development in a given time frame and/or reduce the extent of the disease in a given time frame, when compared to not using the method. In some aspects, such comparisons are based on clinical studies using a statistically significant number of Subjects. Disease development can be detectable using standard clinical techniques. Development may also refer to disease progression that can be initially undetectable and includes occurrence, recurrence, and onset.

As used herein “palliating pain” (e.g., pain associated with an inflammatory disease such as a type of arthritis) or “one or more symptoms of a pain” refers to lessening the extent of one or more undesirable clinical manifestations of pain in veterinary species treated with a composition of GMSC in accordance with the invention.

The terms “reducing”, “suppressing” and “inhibiting” have their commonly understood meaning of lessening or decreasing.

The term “transplanted”, as used herein, refers to transferring GMSC alone (genetically modified or not) or GMSC (genetically modified or not) or their conditioned media or extracellular vesicles that are embedded in a support matrix, media, polymer, gel or any other excipients into a patient's body. The cells can be autologous, allogeneic, or xenogeneic.

As used herein, the term “xenogeneic” or, “xenogenic transplantation” or “xenogeneic treatment” or “xenogeneic injection” refers to transferring GMSC from one species alone (genetically modified or not) or their conditioned media or extracellular vesicles that are embedded in a support matrix, media, polymer, gel or any other excipients into a patient's body from other species. In one aspect, the transplantation is xenotransplantation in which where the xenotransplantation does not result in any significant rejection of the composition.

As used herein the term “allogeneic” or, “allogeneic transplantation” or “allogeneic treatment” or “allogeneic injection” refers to transferring GMSC from one species alone (genetically modified or not) or their conditioned media or extracellular vesicles that are embedded in a support matrix, media, polymer, gel or any other excipients into a patient's body from the same species.

As used herein the term “autologous” or, “autologous transplantation” or “autologous treatment” or “autologous injection” refers to transferring GMSC from one species alone (genetically modified or not) or their conditioned media or extracellular vesicles that are embedded in a support matrix, media, polymer, gel or any other excipients into a patient's body from the same species

As used herein, the term “infectious diseases” refers to chronic infectious diseases, subacute infectious diseases, acute infectious diseases, viral diseases, bacterial diseases, protozoan diseases, parasitic diseases, fungal diseases, mycoplasma diseases and prion diseases.

As used herein, the term “autoimmune disorders” or “autoimmune diseases” or “autoimmune conditions” refers to autoimmune cardiovascular diseases, rheumatoid diseases, autoimmune glandular diseases, autoimmune gastrointestinal diseases, autoimmune cutaneous diseases, autoimmune hepatic diseases, autoimmune neurological diseases, autoimmune muscular diseases, autoimmune nephric diseases, autoimmune diseases related to reproduction, autoimmune connective tissue diseases and autoimmune systemic diseases. Examples of autoimmune cardiovascular diseases include, but are not limited to atherosclerosis, myocardial infarction, thrombosis, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing and crescentic glomerulonephritis, antiphospholipid syndrome, antibody-induced heart failure, thrombocytopenic purpura, autoimmune hemolytic anemia, cardiac autoimmunity in Chagas' disease and anti-helper T lymphocyte autoimmunity. Examples of autoimmune rheumatoid diseases include rheumatoid arthritis and ankylosing spondylitis. Examples of autoimmune glandular diseases include pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include autoimmune diseases of the pancreas, Type 1 diabetes, autoimmune thyroid diseases, Graves' disease, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Examples of autoimmune gastrointestinal diseases include chronic inflammatory intestinal diseases, celiac disease, colitis, ileitis and Crohn's disease. Examples of autoimmune cutaneous diseases include autoimmune bullous skin diseases, such as, but are not limited to, pemphigus vulgaris, bullous pemphigoid and pemphigus foliaceus. Examples of autoimmune hepatic diseases include hepatitis, autoimmune chronic active hepatitis, primary biliary cirrhosis (and autoimmune hepatitis. Examples of autoimmune neurological diseases include, but are not limited to, multiple sclerosis, Alzheimer's disease, myasthenia gravis, neuropathies, motor neuropathies; Guillain-Barre syndrome and autoimmune neuropathies, myasthenia, Lambert-Eaton myasthenic syndrome; paraneoplastic neurological diseases, cerebellar atrophy, paraneoplastic cerebellar atrophy and stiff-man syndrome; non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies; dysimmune neuropathies; acquired neuromyotonia, arthrogryposis multiplex congenita, neuritis, optic neuritis and neurodegenerative diseases. Examples of autoimmune muscular diseases include, but are not limited to, myositis, autoimmune myositis and primary Sjogren's syndrome and smooth muscle autoimmune disease. Examples of autoimmune nephric diseases include nephritis and autoimmune interstitial nephritis. Examples of autoimmune diseases related to reproduction include, but are not limited to, repeated fetal loss. Examples of autoimmune connective tissue diseases include ear diseases, autoimmune ear diseases and autoimmune diseases of the inner ear. Examples of autoimmune systemic diseases include systemic lupus erythematosus and systemic sclerosis.

Representative examples of CNS diseases or disorders that can be beneficially treated with the cells described herein include, but are not limited to, a pain disorder, a neurodegenerative disease or disorder and a convulsive disorder. More specific examples of such conditions include Parkinson's, ALS, Multiple Sclerosis, Huntingdon's disease, autoimmune encephalomyelitis, diabetic neuropathy, glaucomatous neuropathy, macular degeneration, action tremors and tardive dyskinesia, panic, anxiety, depression, alcoholism, insomnia, manic behavior, Alzheimer's and epilepsy.

As used herein, the term “graft rejection” refers to graft rejection, chronic graft rejection, subacute graft rejection, hyperacute graft rejection, acute graft rejection and graft versus host disease.

As used herein, the term “allergic conditions or diseases” refers to asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

As used herein, the term “inflammatory” or “inflammatory disorders” or “inflammatory diseases” refers to chronic inflammatory diseases and acute inflammatory diseases. In some embodiments the inflammatory disease or condition is associated with hypersensitivity.

Inflammatory Diseases Associated with Hypersensitivity Examples of hypersensitivity include Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH. Type I or immediate hypersensitivity, such as asthma. Type II hypersensitivity include, but are not limited to, rheumatoid diseases, rheumatoid autoimmune diseases, rheumatoid arthritis, spondylitis, ankylosing spondylitis, systemic diseases, systemic autoimmune diseases, systemic lupus erythematosus, sclerosis, systemic sclerosis, glandular diseases, glandular autoimmune diseases, pancreatic autoimmune diseases, diabetes, Type I diabetes, thyroid diseases, autoimmune thyroid diseases, Graves' disease, thyroiditis, spontaneous autoimmune thyroiditis, Hashimoto's thyroiditis, myxedema, idiopathic myxedema; autoimmune reproductive diseases, ovarian diseases, ovarian autoimmunity, autoimmune anti-sperm infertility, repeated fetal loss, neurodegenerative diseases, neurological diseases, neurological autoimmune diseases, multiple sclerosis, Alzheimer's disease, myasthenia gravis, motor neuropathies, Guillain-Barre syndrome, neuropathies and autoimmune neuropathies, myasthenic diseases, Lambert-Eaton myasthenic syndrome, paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies; neuropathies, dysimmune neuropathies; neuromyotonia, acquired neuromyotonia, arthrogryposis multiplex congenita, cardiovascular diseases, cardiovascular autoimmune diseases, atherosclerosis, myocardial infarction thrombosis, granulomatosis, Wegener's granulomatosis, arteritis, Takayasu's arteritis and Kawasaki syndrome; anti-factor VIII autoimmune disease; vasculitises, necrotizing small vessel vasculitises, microscopic polyangiitis, Churg and Strauss syndrome, glomerulonephritis, pauci-immune focal necrotizing glomerulonephritis, crescentic glomerulonephritis; antiphospholipid syndrome; heart failure, agonist-like .beta.-adrenoceptor antibodies in heart failure, thrombocytopenic purpura; hemolytic anemia, autoimmune hemolytic anemia, gastrointestinal diseases, autoimmune diseases of the gastrointestinal tract, intestinal diseases, chronic inflammatory intestinal disease, celiac disease (autoimmune diseases of the musculature, myositis, autoimmune myositis, Sjogren's syndrome; smooth muscle autoimmune disease, hepatic diseases, hepatic autoimmune diseases, autoimmune hepatitis and primary biliary cirrhosis. Type IV or T cell mediated hypersensitivity, include rheumatoid diseases, rheumatoid arthritis, systemic diseases, systemic autoimmune diseases, systemic lupus erythematosus, glandular diseases, glandular autoimmune diseases, pancreatic diseases, pancreatic autoimmune diseases, Type 1 diabetes; thyroid diseases, autoimmune thyroid diseases, Graves' disease; ovarian diseases, prostatitis, autoimmune prostatitis, polyglandular syndrome, autoimmune polyglandular syndrome, Type I autoimmune polyglandular syndrome, neurological diseases, autoimmune neurological diseases, multiple sclerosis, neuritis, optic neuritis, myasthenia gravis, stiff-man syndrome, cardiovascular diseases, cardiac autoimmunity in Chagas' disease, autoimmune thrombocytopenic purpura, anti-helper T lymphocyte autoimmunity, hemolytic anemia, hepatic diseases, hepatic autoimmune diseases, hepatitis, chronic active hepatitis, biliary cirrhosis, primary biliary cirrhosis, nephric diseases, nephric autoimmune diseases, nephritis, interstitial nephritis, connective tissue diseases, ear diseases, autoimmune connective tissue diseases, autoimmune ear disease, disease of the inner ear, skin diseases, cutaneous diseases, dermal diseases, bullous skin diseases, pemphigus vulgaris, bullous pemphigoid and pemphigus foliaceus. Examples of delayed type hypersensitivity include contact dermatitis and drug eruption. Examples of types of T lymphocyte mediating hypersensitivity include, but are not limited to, helper T lymphocytes and cytotoxic T lymphocytes. Examples of helper T lymphocyte-mediated hypersensitivity include, but are not limited to, T.sub.h1 lymphocyte mediated hypersensitivity and T.sub.h2 lymphocyte mediated hypersensitivity.

As used herein, the term “culture media” or “basal media” or “growth media” refers to refers to a solution of amino acids, vitamins, salts, and nutrients that is effective to support the growth of cells in culture, although normally these compounds will not support cell growth unless supplemented with additional compounds. In a non-limiting example, the nutrients can include a carbon source (e.g., a sugar such as glucose) that can be metabolized by the cells, as well as other compounds necessary for the cells' survival. A number of growth media are known in the art of mammalian cell culture, such as Dulbecco's Modified Eagle Media (DMEM), Knockout-DMEM (KO-DMEM), DMEM/F12, although any basal media that can be supplemented with molecules that can support cells proliferation, differentiation or production of bioactive agent.

As used herein “rapid adherence to culture plastic” refers to cells that adhere between 2-12 h after the initial plating.

As used herein, “culture plastic” refers to the cells that are cultured in plastic flasks, plastic dishes, plastic bottles and the like.

As used herein, the term “cell differentiation” refers to the ability of the stem cell to commit to a cell lineage, in a not limiting example cell differentiation into chondrogenic lineages.

Disclosed embodiments comprise therapeutic use of “induction of GMSC biofactors”. For example, embodiments comprise adding specific supplements in the GMSC's culture media, in order to maximize their therapeutic benefit for specific uses. in a non-limiting examples, in the context of GMSC refers to the use of stimulatory agents including, for example, inflammatory cytokines or mediators (non limiting examples: IFN-γ, TNF-α, IL-1β, FGF), hypoxia, pharmacological drugs and chemical agents (non limiting examples: valproic acid, sphingosine-1-phosphate, 5-aza-2′-deoxycytidine, desferrioxamine, 2,4-dinitrophenol, dimethyloxalylglycine, isoflurane, 2-chloro-N6-cyclopentyl-adenosine), biomaterials and different culture conditions (non limiting examples: 3D cell culture in collagen-hydrogel scaffold or chitosan scaffold, 3D cell culture in hydrogel, Encapsulation in hydrogel) and combinations thereof to enhance GMSC function. GMSC are exposed to inflammatory cytokines to increase expression of adhesion molecules such as VCAM-1 and ICAM-1, which enables MSC to sequester and enhance function of immune cells.

As used herein, the term “bioactive agents” or “biofactors” refers to any organic, inorganic, or living agent that is biologically active or relevant. It can also include a man-made particle or material, which carries a biologically relevant or active material. An example is a nanoparticle comprising a core with a drug and a coating on the core. Bioactive agents also can include drugs such as chemical or biological compounds that can have a therapeutic effect on a biological organism. Non-limiting examples include a protein, a polypeptide, a polysaccharide (e.g. heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor inhibitor, a growth factor receptor, an anti-inflammatory agent, an antimetabolite, an integrin blocker, or a complete or partial functional in sense or antisense gene, growth factors, anti-rejection agents, anti-inflammatory agents, anti-infective agents (e.g., antibiotics and antiviral agents), analgesics and analgesic combinations, anti-asthmatic agents, anticonvulsants, antidepressants, anti-diabetic agents, anti-neoplastics, anticancer agents, anti-psychotics, antioxidants, immunosuppressive agents, vitamins and minerals, and agents used for cardiovascular diseases such as anti-restenosis and anti-coagulant compounds. Bioactive agents also can include precursor materials that exhibit the relevant biological activity after being metabolized, broken-down (e.g. cleaving molecular components), or otherwise processed and modified within the body. These can include such precursor materials that might otherwise be considered relatively biologically inert or otherwise not effective for a particular result related to the medical condition to be treated prior to such modification. Combinations, blends, or other preparations of any of the foregoing examples can be made and still be considered bioactive agents within the intended meaning herein. Aspects of the present disclosure directed toward bioactive agents can include any or all of the foregoing examples. In one embodiment, GMSC may secrete bioactive agents and they can be used as conditioned media, extracellular vesicles and/or exosomes.

As used herein “immunosuppressive agent” refers to any agent that prevents, delays the occurrence of, or decreases the intensity of, the desired immune response, e.g., rejection of a transplanted cell, tissue, or organ. In one embodiment, the bioactive agent is an immunosuppressive agent. In one embodiment, GMSC may secrete bioactive agents and they can be used as conditioned media, extracellular vesicles and/or exosomes.

As used herein, the term “conditioned media” refers to a growth media that is further supplemented with soluble factors (“culture-derived growth factors”) derived from GMSC, cultured in the media. In some embodiments the conditioned media is growth media conditioned by the growth of GMSC. Techniques for isolating conditioned media from a cell culture are known in the art. In some embodiments of the invention, the conditioned media is essentially cell-free. In this context, “essentially cell-free” refers to a conditioned media that contains fewer than about 10%, fewer than about 5%, 1%, 0.1%, 0.01%, 0.001%, and 0.0001% than the number of cells per unit volume, as compared to the culture from which it was separated. As used herein, the term “conditioned media” also encompasses such a media that has been treated by concentration, filtration, extraction, fractionation or other means for preserving, increasing the potency, improving the stability, removing impurities, etc.

As used herein, the term “extracellular vesicles” refers to extracellular vesicles that are membrane surrounded structures released or secreted by mesenchymal stromal cells GMSC that are cultured in a culture media that is essentially free of vesicles. The extracellular vesicles may include exosomes, microvesicles, cell-derived vesicles, and shedding vesicles.

As used herein, the term “not triggering” or “suppression” of the immune response, will be understood by those ordinary skill in the art that this does not requires that the immune system of the host lies absolutely dormant, but merely that any acidity of immune system with respect to the transplanted cells does not rise to a level that prevents, suppresses, pr negates the action of the transplanted cells.

As used herein, the term “to enhance treatment” refers to helping the patient to recover from its conditions. It can be any technique that may be implemented, in a not limiting example a cell engineering method aiming to upregulate anti-inflammatory molecules, cell engineering method aiming to downregulate pro-inflammatory molecules, a cell differentiation induction. Additions include excipient solution, hyaluronic acid and the like.

As used herein, the term “genetically modified cell”, “modified cell”, “transgenic cell” refers to cells that are genetically modified outside the body in order to enhance the patient's therapy. In non-limiting examples a new gene can be introduced, faulty gene can be corrected in the cells.

As used herein, “immunosuppression” refers to prevention of the immune response (for example by the administration of an “immunosuppresive agent”, as defined herein) such that an “immune response”, as defined herein, is not detectable. As used herein, “prevention” of an immune response means an immune response is not detectable. An immune response (for example, transplant rejection or antibody production) is detected according to methods well-known in the art and defined herein. “Immunosuppression” also means a delay in the occurrence of the immune response as compared to any one of a transplant recipient that has not received an immunosuppresive agent, or a transplant recipient that has been transplanted with material that is not “immunologically blinded” or “immunoprivileged”, as defined herein. A delay in the occurrence of an immune response can be a short delay, for example 1 hr-10 days, i.e., 1 hr, 2, 5 or 10 days. A delay in the occurrence of an immune response can also be a long delay, for example, 10 days-10 years (i.e., 30 days, 60 days, 90 days, 180 days, 1, 2, 5 or 10 years). “Immunosuppression” also means a decrease in the intensity of an immune response. The intensity of an immune response can be decreased such that it is 5-100%, preferably, 25-100% and most preferably 75-100% less than the intensity of the immune response of any one of a transplant recipient that has not received an immunosuppresive agent, or a transplant recipient that has been transplanted with material that is not autologous. The intensity of an immune response can be measured by determining the time point at which transplanted material is rejected. The intensity of an immune response can also be measured by quantitating the amount of a particular antibody capable of binding to the transplanted material, wherein the level of antibody production correlates directly with the intensity of the immune response. Alternatively, the intensity of an immune response can be measured by determining the time point at which a particular antibody capable of binding to the transplanted material is detected.

An “effective amount” (when used in the treatment or prophylaxis context, or in the context of palliating pain or alleviating the symptoms of a particular condition) refers to an amount sufficient to effect beneficial or desired results including clinical results. An effective amount can be administered in one or more administrations. For purposes of this invention, an effective amount of GMSC is a certain amount of cells that can reduce one or more symptoms of the conditions for which the animal or human is being treated. For example, reduction of limping in an animal to whom GMSC has been administered is one symptom for arthritis in a non-human animal and observation of this reduction of this particular symptom could mean that an effective amount of GMSC was given to the animal. In one aspect, the GMSC are cultured further to induce them to differentiate down a particular pathway. In another aspect, the GMSC are cultured in a manner where no differentiation occurs.

As used herein, the term “excipient” refers to an inert substance added to a pharmaceutical composition to further facilitate administration of an active ingredient. Examples, without limitation, of excipients include Phosphate Buffer Solution (PBS), calcium carbonate, calcium phosphate, various sugars and types of starch, cellulose derivatives, gelatin, vegetable oils, and polyethylene glycols.

“Systemically” routes of administration or “systemic injection” may, in non-limiting examples, include oral, rectal, transmucosal, especially transnasal, intestinal, or parenteral delivery, including intramuscular, subcutaneous, and intramedullary injections, as well as intrathecal, direct intraventricular, intravenous, intraperitoneal, intranasal, or intraocular injections.

Alternatively, one may administer the pharmaceutical composition in a local rather than systemic manner, for example, via injection of the pharmaceutical composition directly into a tissue region of a patient.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

As used herein “pharmaceutically acceptable carrier” refers to any material which, when combined with an active ingredient, allows the ingredient to retain biological activity and does not provoke an unacceptable immune response (e.g., a severe allergy or anaphylactic shock) based on the knowledge of a skilled practitioner. Examples include any of the standard pharmaceutical carriers such as carboxymethylcellulose (CMC), phosphate buffered saline solutions, water, hyaluronic acid, emulsions such as oil/water emulsion, and various types of wetting agents. Exemplary diluents for aerosol or parenteral administration are phosphate buffered saline or normal (0.9%) saline. An exemplary carrier for the infusion of cells is CMC. Compositions comprising Such carriers are formulated by well-known conventional methods.

General reference to “the composition” or “compositions” includes and is applicable to compositions of the invention. The invention also provides pharmaceutical compositions comprising the components described herein. As used herein, the singular form “a”, “an”, and “the” includes plural references unless indicated otherwise. For example, “an GMSC includes one or more adipose tissue derived stem cells. Reference to “about a value or parameter herein includes (and describes) aspects that are directed to that value or parameter perse. For example, description referring to “about X” includes description of “X” It is understood that aspects and aspects of the invention described herein include “comprising” “consisting, and “consisting essentially of aspects and aspects.

Conditioned media, concentrated conditioned media, extracts and fractions, extracellular vesicles and exosomes derived from GMSC thereof can be used by formulators for preparing compositions comprising cosmetic, cosmeceutical, or pharmaceutical formulations with cosmetically-acceptable, cosmeceutically-acceptable or pharmaceutically-acceptable carriers. The skilled artisan will appreciate that cosmetically-acceptable carriers, cosmeceutically-acceptable carriers and pharmaceutically-acceptable carriers may be the same or different, depending on the intended application of the composition.

As used herein, the term “cosmeceutical” refers to a formulation or composition comprising at least one biologically active ingredient that has an effect on the user of the product and at least one cosmeceutically-acceptable carrier. Cosmeceuticals may be viewed as cosmetics that, in certain applications and under appropriate conditions, may provide medicinal or drug-like benefits. In certain applications, for example, cosmeceuticals may affect the underlying structure of the skin, decrease wrinkle depth, or reverse or ameliorate the effect of photooxidation or aging on the skin. Cosmeceuticals may be particularly useful as skin care products, hair care products, and sun care products. In certain embodiments, cosmeceutical compositions comprise delivery systems including at least one of liposomes, cyclodextrins, polymer systems, or hyaluronic acid or related compounds. Cosmeceutical compositions comprise cosmeceutically-acceptable carriers. The skilled artisan will understand that a pharmaceutically-acceptable carrier or formulation that is suitable for topical applications will typically also be a cosmeceutically-acceptable carrier or formulation.

A topical cosmetic or cosmeceutical ointment, lotion, or gel composition typically contains an effective amount of conditioned media or extracts thereof and may comprise other active and inert ingredients as well in a cosmetically- or a cosmeceutically-acceptable carrier, such as a pharmaceutical cream base, an oil-in-water emulsion, a water-in-oil emulsion, a gel, or the like. Various cosmetic and cosmeceutical compositions for topical use include drops, tinctures, lotions, creams, salves, serums, solutions, and ointments containing conditioned media or extracts, and an appropriate carrier. The optimal percentage of the conditioned media or extract in each composition varies according to the composition's formulation and the therapeutic effect desired.

The skilled artisan in the formulation arts will understand that the inventive compositions may comprise any of a number of cosmetically-, cosmeceutically-, or pharmaceutically-acceptable formulations, depending on the type of product, the nature of the composition, the location of composition's use, the desired effect, and the like.

While proprietary formulations are common in the formulation arts, formulators of ordinary skill will be able to determine or readily select appropriate formulations for specific applications without undue experimentation.

Compositions of the present invention may, if desired, be presented in a pack or dispenser device, such as an FDA-approved kit, which may contain one or more unit dosage forms containing the active ingredient (e.g., cells). The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

### How GMSC Work

Gonadal-Derived Stem Cells and their Isolation ovarian and testicular tissue offers a source of stromal cells.

Gonadal tissue is readily accessible and abundant in many subjects (e.g., non-human mammals, veterinary species and even humans). The fact that the testis is always forming new cells and the ovaries are naturally healed after the ovulation wound injury, suggests that these tissues contain stromal stem cells that are capable of self-renewal. Methods for the ordinary isolation, expansion, and differentiation of mesenchymal tissue-derived stem cells have been described previously, however the technique of purification and isolation from the ovaries was first reported by the author of this invention. These techniques are equally applicable to the isolation, expansion, and differentiation of any veterinary species and human GMSC.

GMSC may be isolated from any veterinary species and human, including monkey, baboon, chimpanzee, orangutan, panda, tiger, lion, bear, cheetah, llama, turtles, birds, snakes, mice, rats, pigs, cows, horses, goats, sheep, dogs, cats, rabbits, ferrets and the like.

In one aspect, a Subject from whom GMSC can be isolated and used for therapeutic and/or prophylactic purposes is a porcine species. Porcine species are commonly raised for purpose of providing parts for Xenotransplantation (e.g., valve for human heart). Accordingly, porcine GMSC may be used for Xenotransplantation into other veterinary species and even humans. In another aspect, the invention provides for GMSC obtained from privately owned pets, whose procurement may be regulated by the Food and Drug Administration (FDA). The GMSC from these pets may be used for Xenotransplantation, allogeneic transplantation or autologous transplantation. In one aspect, the invention provides for GMSC that are xenogeneic to the recipient of the GMSC-based treatment. Non-limiting examples of types of GMSC that can be used are bovine, equine, ovine and porcine. In another aspect, the invention provides for GMSC that are allogeneic to the recipient of the GMSC based treatment. In another aspect, the invention provides for GMSC that are autologous to the recipient of the GMSC-based treatment. In another aspect, the invention provides for GMSC that are autologous to the recipient of the GMSC-based treatment.

GMSC from veterinary species may be isolated in any way known to one of skill in the art. In one aspect of the present invention, GMSC may be isolated according to the following non-limiting method. First, gonadal tissue is collected after castration surgery, biopsy, or in slaughterhouses, the tissue is kept on ice until arrival at the lab in any buffer solution containing antimicrobials, in non limited example solution with Phosphate Buffer Solution (PBS) containing 1% penicillin and streptomycin, minced into small pieces, then mixed with a solution with one or more dissociation enzymes selected from the group consisting of collagenase (any type), dispase, trypsin, papain, hyaluronidase, elastase, DNase, pronase, pepsin, thermolysin, or combinations thereof, at a concentration ranging from about 0.001% to about 10% (w/v), optionally in the presence of chelating agents, salts, or buffers. The tissue is incubated with said enzyme solution for a period of about 20 minutes to about 24 hours, at a temperature ranging from about 4° C. to about 38° C., under statis or agitated conditions, wherein the incubation time and temperature are selected according to the enzyme or enzyme mixture used. In a non limiting example collagenase at 37 C for 2 hours. Centrifugation is performed at speeds between about 50×g and about 20,000×g, for a period of about 1 minute to about 2 hours, at a temperature between about 4° C. and about 38° C. The pellet is recovered and resuspended in cell culture media for adherent cells considering the following but not only: DMEM, alpha-MEM, MEM, DMEM F12, supplement with bovine calf serum or in the absence of it, with: knockout serum replacement (same concentrations) considering the following but not only concentrations from 30% and 1% and with 1% of antimicrobial agent. The tube containing the resuspended cell with any of the media composition listed above, is centrifuge at 220×g for 10 minutes at room temperature. The pellet is resuspended, and the cells are plated into any adherent cell culture dish. The selection for cell purity is done by first removal of the media at least 2 h after the initial plating. In one non-limiting aspect, the selection can also be done until 5 h of the initial plating. The selection can also be done until 24 h of the initial plating. This method is based on the selective capabilities of cells to rapidly adhere into the culture dish. The non-adherent cells (those that do not attach in the first 5 h of culture) will be removed from the culture dish allowing the most adherent cells to be isolated. This method facilitated the development of homogenous cell populations, without the necessity of expensive techniques, such as FACS. Following isolation of those cells, they can be cultured, expanded, differentiated, studied further, or administered to subjects in one or more of the methods of treatment disclosed herein.

In embodiments, GMSC can be activated to produce desired characteristics. For example, GMSC can be polarized towards a pro- or anti-inflammatory phenotype depending on the Tol-like receptor (TLR) stimulated. In a non-limiting example, GMSC are exposed to inflammatory cytokines to increase expression of adhesion molecules such as VCAM-1 and ICAM-1, which enables MSC to sequester and enhance the function of immune cells.

Disclosed formulations can be provided as a kit. For example, a disclosed kit can comprise a pharmaceutically acceptable carrier; an isolated population of mesenchymal stem cells; isolated interferons, isolated interleukins, and instructions for using the kit in a method for attenuating an immune response. The cell and cytokine components of the kit can be administered individually, or combined in vitro and subsequently administered as a mixture. The kit also optionally may include a means of administering the composition, for example by injection.

Disclosed formulations comprise MSC combined with cytokines in the form of a composition, e.g., a pharmaceutical composition suitable for administration to a subject in need of treatment with the same.

In one aspect of the present invention, GMSC (genetically modified or not, activated or not) conditioned media may be isolated according to the following non-limiting method. The culture media with supplements of GMSC can be removed and new media containing only the basal media and antimicrobiotics agents is added. After 24 h to 7 days, the media can be removed and centrifuged. The supernatant should be recovered and filtered, using a 0.22 micron filter. For certain applications the filtered media can be concentrated, for example, by ultrafiltration (suitable cross flow hollow fiber ultrafiltration cartridges are available from A/G Technology Corp., Needham, Mass.) or ultracentrifugation, or density gradients for extracellular vesicles and/or exosomes isolation. After that, the conditioned media, extracellular vesicles and/or exosomes can be stored (lyophilized or not), in a non-limiting example in minus four degrees, or used immediately.

In one aspect of the present invention, in some cases, freshly-thawed cells were prepared for therapeutic use. Frozen cells were thawed in an automated thawing system and after removal of the cryomedia, cells are tested for viability and sterility and re-suspended in an injectable solution in a sterile syringe and used in the patient.

The invention also provides for therapeutic compositions which are useful in practicing the therapeutic methods of this invention. In some embodiments, the GMSC used are Xenogeneic to the recipient. In another aspect, the GMSC used are allogeneic to the recipient. In another aspect, the GMSC used are autologous to the recipient. The invention also provides for a bank of GMSC which can be used as a “universal donor” for treatment (therapeutic or prophylactic) for any veterinary species and even humans. In one aspect of the invention, the bank of GMSC can also be done for autologous use. The invention also provides for a therapeutic composition includes, in admixture, a pharmaceutically acceptable excipient (carrier) or media and the GMSC of the present invention, including cells or tissues derived therefrom, alone or in combination with one or more bioactive agents, and at a strength effective for administration by various means to a subject experiencing cellular or tissue loss or deficiency. In another aspect, the present invention provides for therapeutic compositions for use in methods which comprise or are based upon the GMSC of the present invention, including lineage-uncommitted populations of cells, lineage-commit ted populations of cells or tissues derived therefrom, along with a pharmaceutically acceptable carrier or media. It is to be understood that the invention also encompasses therapeutic compositions comprising bioactive agents that act on or modulate the GMSC of the present invention and/or the cells or tissues derived therefrom, along with a pharmaceutically acceptable carrier or media. The preparation of cellular or tissue-based therapeutic compositions is well understood in the art. Such compositions may be formulated in a pharmaceutically acceptable media. The cells may be in solution or embedded in a matrix (natural or synthetic) or gels, including but not limited to Hyaloronic acid hydrogel.

The preparation of therapeutic compositions with bioactive agents (for example, growth factors) as active ingredients is well understood in the art. The active therapeutic ingredient is often mixed with excipients or media which are pharmaceutically acceptable and compatible with the active ingredient. In addition, if desired, the composition can contain minor amounts of auxiliary substances such as wetting or emulsifying agents, pH buffering agents which enhance the effectiveness of the active ingredient. A bioactive agent can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, sodium, potassium, ammonium, calcium, or ferric hydroxides, and such organic bases as isopropylamine, trimethylamine, 2-ethylamino ethanol, histidine, procaine, and the like.

The therapeutic compositions of the present invention are administered in a manner compatible with the dosage formulation, and in a therapeutically effective amount. The quantity to be administered depends, for instance, on the Subject and debilitation to be treated. However, suitable dosages of the therapeutic composition of the present invention may range from about 0.05-100.0 million GMSC. Suitable regimens for initial administration and follow on administration are also variable, but can include an initial administration followed by repeated doses at one or more intervals as desired or indicated (e.g. weeks, months, or years) by a subsequent injection or other administration. One of skill in the art may readily determine the appropriate concentration of GMSC for a particular purpose. An exemplary dose is in the range of about 0.05-100 million GMSC per treatment site per day. In a non-limiting example, approximately 4 million GMSC are injected into a veterinary patient (e.g., a stiff joint) to treat osteoarthritis. Precise administration schedules for the therapeutic composition depend on the judgment of the veterinarian and the desired result and are therefore peculiar, to a certain extent, to each Subject. The GMSC or differentiated cells of the present invention can be administered by injection into a target site of a subject, preferably via a delivery device, such as a tube, e.g., catheter. In one aspect, the tube additionally contains a needle, e.g., a syringe, through which the GMSC can be introduced into the subject at a desired location. Specific, non-limiting examples of administering cells to Subjects may also include administration by subcutaneous injection, intramuscular injection, intraarticular, epidural or intravenous injection. If administration is intramuscular, an injectable liquid suspension of cells can be prepared and administered into the patient's muscle. In another aspect, if the medical condition to be treated is dilated cardiomyopathy, then one of skill in the art (e.g., a veterinarian) can deliver GMSC using catheter-based injection into the coronary artery or in a manner such that the GMSC are trapped in capillary beds so that they can be distributed to Surrounding tissue. Cells may also be inserted into a delivery device, e.g., a syringe, catheter, in different forms. For example, the cells can be suspended in a solution contained in such a delivery device.

As used herein, the term “solution” includes a pharmaceutically acceptable carrier or diluent in which the GMSC of the invention remain viable. The use of such carriers and diluents is well known in the art. The solution is preferably sterile and fluid to the extent that easy syringe ability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating GMSC or their differentiated cells as described herein, in a pharmaceutically acceptable carrier or diluent and, as required, other ingredients enumerated above, followed by filter sterilization. The GMSC may be administered systemically (for example intravenously) or locally (for example directly into a myocardial defect under echocardiogram guidance, or by direct application under visualization during surgery). For such injections, the GMSC or their differentiated cells may be in an injectable liquid suspension preparation or in a biocompatible media which is injectable in liquid form and becomes semi-solid at the site of damaged tissue. A conventional intra-cardiac syringe or a controllable endoscopic delivery device can be used so long as the needle lumen or bore is of sufficient diameter (e.g., 30 gauge or larger) that shear forces will not damage the cells being delivered. Cells may be administered in any manner that permits them to graft to the intended tissue site and reconstitute or regenerate the functionally deficient area. Support matrices into which the GMSC or their differentiated cells can be incorporated or embedded include matrices which are biocompatible, recipient-compatible and which degrade into products which are not harmful to the recipient. These matrices provide support and protection for GMSC or their differentiated cells in vivo. Natural and/or synthetic biodegradable matrices are examples of such matrices. Natural biodegradable matrices include plasma clots, e.g., derived from a mammal, collagen, fibronectin, and laminin matrices. Suitable synthetic material for a cell transplantation matrix must be biocompatible to preclude migration and immunological complications; and should be able to support extensive cell growth and differentiated cell function. It must also be resorbable, allowing for a completely natural tissue replacement. The matrix should be configurable into a variety of shapes and should have sufficient strength to prevent collapse upon implantation. A variety of studies indicate that the biodegradable polyester polymers made of polyglycolic acid fulfill all of these criteria. Other synthetic biodegradable Support matrices include synthetic polymers such as polyanhydrides, polyorthoesters, and polylactic acid. Further examples of synthetic polymers and methods of incorporating or embedding cells into these matrices are also known in the art. Attachment of the cells to the polymer may be enhanced by coating the polymers with compounds such as basement membrane components, agar, agarose, gelatin, gum arabic, collagens types I, II, III, IV and V, fibronectin, laminin, glycosaminoglycans, mixtures thereof, and other materials known to those skilled in the art of cell culture. All polymers for use in the matrix must meet the mechanical and biochemical parameters necessary to provide adequate support for the cells with subsequent growth and proliferation. One of the advantages of a biodegradable polymeric matrix is that angiogenic and other bioactive compounds can be incorporated directly into the support matrix so that they are slowly released as the support matrix degrades in vivo. As the cell-polymer structure is vascularized and the structure degrades, GMSC may differentiate according to their inherent characteristics. Factors, including nutrients, growth factors, inducers of differentiation or de-differentiation (i.e., causing differentiated cells to lose characteristics of differentiation and acquire characteristics such as proliferation and more general function), products of secretion, immunomodulators, inhibitors of inflammation, regression factors, bioactive agents which enhance or allow ingrowth of the lymphatic network or nerve fibers, hyaluronic acid, and drugs, which are known to those skilled in the art and commercially available with instructions as to what constitutes an effective amount, from suppliers such as Collaborative Research, Sigma Chemical Co., Vascular growth factors such as vascular endothelial growth factor (VEGF), epidermal growth factor (EGF), and heparin binding epidermal growth factor like growth factor (HB-EGF), could be incorporated into the matrix or provided in conjunction with the matrix. Similarly, polymers containing peptides such as the attachment peptide RGD (Arg-Gly-Asp) can be synthesized for use in forming matrices. In another example, the cells may be transplanted in a gel matrix (such as Gelfoam from Upjohn Company) which polymerizes to form a substrate in which GMSC or their differentiated cells can grow. A variety of encapsulation technologies have been developed. PLGA or poly(lactic-co-glycolic acid) is a Food and Drug Administration (FDA)-approved copolymer which is used in a host of therapeutic devices, owing to its biodegradability and biocompatibility. PLGA is synthesized by means of random ring-opening co-polymerization of two different monomers, the cyclic dimers (1,4-dioxane-2,5-diones) of glycolic acid and lactic acid. Common catalysts used in the preparation of this polymer include tin(II) 2-ethylhexanoate, tin(II) alkoxides, or aluminum isopropoxide. During polymerization, Successive monomeric units (of glycolic or lactic acid) are linked together in PLGA by ester linkages, thus yielding alinear, aliphatic polyester as a product. PLGA has been successfully used as a biodegradable polymer because it undergoes hydrolysis in the body to produce the original monomers, lactic acid and glycolic acid. These two monomers are by-products of various metabolic pathways in the body. Since the body is able to effectively break down the two monomers, there is no systemic toxicity associated with using PLGA for drug delivery or biomaterial applications.

In addition, GMSC can be engineered to contain genes that express growth factors, hormones, and cytokines. For example, the GMSC could be engineered to express beneficial genes, such as, without limitation, VEGF, BDNF, IGF, TGF, NGF and other neurotrophic and vasculotrophic growth factors. Injection of a specifically engineered GMSC may help the regeneration of certain tissues; for example, BDNF for nerves. GMSC can also be engineered to express beta cell specific gene Pdx-1, which enable GMSC to secrete insulin. Such cells can be transplanted into Subjects so as to treat their diabetes mellitus. In another aspect, GMSC may be engineered to produce dystrophin and then implanted into Subjects to treat muscular dystrophy. In yet another aspect, GMSC expressing at least one genotypic or phenotypic characteristic of a chondrocyte is genetically modified to express exogenous genes or to repress the expression of endogenous genes and implanted into an animal. In another aspect, GMSC may be engineered to be deficient in histocompatibility complex-I and/or -II. The invention provides a method of genetically modifying Such cells and populations prior to implantation. It is to be understood that GMSC may be able to differentiate further down the lineage towards organ specific cells (e.g., chondrocytes) without the aid of genetic modification. A nucleic acid construct comprising a promoter and the sequence of interest can be introduced into a recipient prokaryotic or eukaryotic cell either as a non-replicating DNA (or RNA) molecule, which can either be a linear molecule or, more preferably, a closed covalent circular molecule. Since such molecules are incapable of autonomous replication without an origin of replication, the expression of the gene can occur through the transient expression of the introduced-sequence. Alternatively, permanent expression can occur through the integration of the introduced DNA sequence into the host chromosome. In one aspect, a vector is employed which is capable of integrating the desired gene sequences into the host cell chromosome. Cells which have stably integrated the introduced DNA into their chromosomes can be selected by also introducing one or more markers which allow for selection of host cells which contain the desired nucleic acid sequence. The marker, if desired, can provide for prototrophy to an auxotrophic host, biocide resistance, e.g., resistance to antibiotics. The selectable marker gene sequence can either be directly linked to the DNA gene sequences to be expressed, or introduced into the same cell by co-transfection. Preferably, expression of the marker can be quantified. In a preferred aspect, the introduced nucleic acid molecule will be incorporated into a plasmid or viral vector capable of autonomous replication in the recipient host. Any of a wide variety of vectors can be employed for this purpose. Factors of importance in selecting a particular plasmid or viral vector include: 1) the ease with which recipient cells that contain the vector can be recognized and selected from those recipient cells which do not contain the vector; 2) the number of copies of the vector which are desired in a particular host; and 3) whether it is desirable to be able to “shuttle the vector between host cells of different species. Preferred eukaryotic vectors include for example, vaccinia virus, SV40, retroviruses, adenoviruses, adeno-associated viruses, lentiviruses and a variety of commercially available, plasmid-based mammalian expression vectors that are familiar to those experienced in the art. Once the vector or nucleic acid molecule containing the construct(s) has been prepared for expression, the DNA construct(s) can be introduced into an appropriate host cell by any of a variety of suitable means, i.e., transformation, transfection, viral infection, conjugation, protoplast fusion, electroporation, particle gun technology, calcium phosphate-precipitation, direct microinjection, and the like. After the introduction of the vector, recipient cells are grown in a selective media, which selects for the growth of vector-containing cells. Expression of the cloned gene molecule(s) results in the production of the heterologous protein. Introduced DNA being “maintained in cells should be understood as the introduced DNA continuing to be present in essentially all of the cells in question as they continue to grow and proliferate. That is, the introduced DNA is not diluted out of the majority of the cells over multiple rounds of cell division. Rather, it replicates during cell proliferation and at least one copy of the introduced DNA remains in almost every daughter cell. Introduced DNA may be maintained in cells in either of two fashions. First, it may integrate directly into the cell's genome. This occurs at a rather low frequency. Second, it may exist as an extrachromosomal element, or episome. In order for an episome not to be diluted out during cell proliferation, a selectable marker gene can be included in the introduced DNA and the cells grown under conditions where expression of the marker gene is required. Even in the case where the introduced DNA has integrated in the genome, a selectable marker gene may be included to prevent excision of the DNA from the chromosome. The genetically modified cells can transiently express the gene of interest or constitutively express the gene of interest. The expression can be either intracellular or on the cell surface. In one aspect of the invention, GMSC of the invention are genetically modified to transiently express one or more cytokines which are beneficial for treatment of a particular medical condition or to palliate pain. For example, GMSC which have been genetically modified to express anti-inflammatory cytokines (e.g., IL-2) can be transplanted into a location where inflammation is occurring (e.g., arthritic joint). The transplantation then provides a dual benefit to the subject because the GMSC can become a chondrocyte in addition to expressing beneficial, anti-inflammatory cytokines. The transient expression can extend for a period of minutes, hours, days or even weeks. In some embodiments of the invention, the transient expression of the beneficial cytokine is 1, 2, or 3 weeks. In other embodiments of the invention, the transient expression of the beneficial cytokine is the length of time necessary for the symptoms to decrease or even disappear. The decrease or disappearance of symptoms can be determined by one's skill in the art (e.g., a veterinarian) who is treating the Subject. The genetically altered cells can then be introduced into the subject by a variety of methods under conditions for the transgene to be expressed in vivo. As a non-limiting example, the transgene can encode for the production of an extracellular matrix protein, preferably wherein the transgene encodes for the production of collagen. The cells containing the transgene for the extracellular matrix protein can then be introduced into the animal. Alternatively, the cells containing the transgene are injected intraperitoneally or into some other Suitable organ depot site.

### GMSC Banks

The invention also provides storage banks of GMSC which have been derived from veterinary species and even humans. Non-limiting examples of types of GMSC that can include all monkey, baboon, chimpanzee, orangutan, panda, tiger, lion, bear, cheetah, llama, turtles, birds, snakes, mice, rats, pigs, cows, horses, goats, sheep, dogs, cats, rabbits, and ferrets.

The banks allow for deposit and/or storage of GMSC which have been isolated from various species. The A GMSC banks allow one of skill in the art (e.g., veterinarian) to treat the animals with ease and with speed. The GMSC banks also allow one of skill in the art to treat the animals with autologous, allogeneic or Xenogeneic GMSC as he/she determines to be appropriate. In one aspect, a bank of veterinary species-derived GMSC which would pass FDA regulation for transplantation is provided for use in veterinary treatments, both therapeutic as well as prophylactic. In another aspect, a bank of porcine-derived GMSC derived from non-FDA approved sources is provided for use in veterinary treatments, both therapeutic as well as prophylactic. In another aspect, the bank of GMSC is allogeneic to the recipient of the treatment. In another aspect, the bank of GMSC is autologous to the recipient of the treatment. In another aspect, the bank of GMSC is xenogeneic to the recipient of the treatment. In another aspect, the bank of GMSC serves as a universal donor to the recipients of veterinary or human treatment.

### GMSC Kits

Kits with GMSC derived from veterinary species also provided are articles of manufacture and kits that include a composition of GMSC derived from any veterinary species and suitable packaging. In one aspect, the composition comprises 100% (referring to purity) GMSC. “Purity” does not refer to the presence of only stem cells in the composition but rather indicates that the stem cells have been manipulated such that they have been removed from their natural tissue environment. Kits comprising GMSC from veterinary species are useful for storage and/or shipment. In some aspects, the invention includes a kit with (i) one or more GMSC or transplantable GMSC derived from one or more veterinary species and (ii) instructions for using the kit to treat a condition in veterinary species in need of such treatment. In various aspects, the invention features a kit with (i) one or more GMSC or transplantable GMSC derived from one or more veterinary species and (ii) instructions for using the kit for research or drug screening uses. In another aspect, the composition of GMSC is combined with different excipients, for example but not limited to hydrogels, matrixes, polymeres. Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. The kit may further comprise a description of selecting an individual suitable or treatment. The containers may be unit doses, bulk packages (e.g., multi-dose packages) or sub-unit doses. For example, kits may also be provided that contain sufficient dosages of GMSC to provide effective treatment for a subject for an extended period. Such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of cells and instructions for use and packaged in quantities Sufficient for storage and use in veterinary clinics, for example, animal hospitals, pharmacies in animal hospitals and supply stores for veterinary clinics and/or animal hospitals. Additionally, the kits may contain compositions comprising GMSC which are Xenogeneic, allogeneic, or autologous to the recipient of the treatment. According to one embodiment, the disease or disorder is selected from the group consisting of a bone or cartilage disease, a neurodegenerative disease, a cardiac disease, a hepatic disease, cancer, nerve damage, wound healing, autoimmune disease, graft versus host disease, spinal cord injury and tissue regeneration.

Bone defects suitable for treatment using the GMSC (genetically modified or not) and/or their derived vesicles, exosomes and conditioned media of the present invention include osteogenesis imperfecta, fracture, congenital bone defects, and the like.

The GMSC (genetically modified or not) and/or their derived vesicles, exosomes and conditioned media of the present invention can be used to treat neurological diseases.

The GMSC (genetically modified or not) and/or their derived vesicles, exosomes and conditioned media of the present invention may be suitable for the treatment of joint conditions including osteoarthritis, rheumatoid arthritis, inflammatory arthritis, chondromalacia, avascular necrosis, traumatic arthritis and the like.

The GMSC (genetically modified or not) and/or their derived vesicles, exosomes and conditioned media can be used to augment engraftment of transplanted hematopoietic or other stem cells and prevent graft-versus-host disease, for example, in bone marrow transplantation or cell transplantation for tissue repair as in implantation for myocardial infarct.

The GMSC (genetically modified or not) and/or their derived vesicles, exosomes and conditioned media of the present invention can be used for the promotion of tissue regeneration. Administration of GMSC (genetically modified or not) and/or their derived vesicles, exosomes and conditioned media has great promise for benefits in regenerative medicine, autoimmune diseases, inflammatory conditions, acute and chronic ischemic conditions reconstructive surgery, tissue engineering, regenerating new tissues and naturally healing diseased or injured organs.

The GMSC (genetically modified or not) and/or their derived vesicles, exosomes and conditioned media of the present invention can be used to treat an inflammatory disease or condition in a patient.

The GMSC (genetically modified or not) and/or their derived vesicles, exosomes and conditioned media may be used for treating autoimmune diseases.

The GMSC (genetically modified or not) and/or their derived vesicles, exosomes and conditioned media can be used for treating infectious diseases.

The GMSC (genetically modified or not) and/or their derived vesicles, exosomes and conditioned media can be used for treating graft rejection.

The GMSC (genetically modified or not) and/or their derived vesicles, exosomes and conditioned media can be used for treating allergic conditions or diseases.

### Methods of Treatment and Administration of GMSC

GMSC from animal derived gonads may be used to treat veterinary species and even humans for urinary incontinence, osteoarthritis, degenerative myelopathy, diabetes, tissue regeneration, wound healing, scarring, soft tissue defect, fecal incontinence, dilated cardiomyopathy, hip dysplasia, avascular necrosis of the femoral head, ligament injury, tendon injury, spinal cord injury, atherosclerosis-related infarctions, arthritis, muscular dystrophy, distemper virus, cognitive dysfunction, dementia, alzheimer, granulomatous meningoencephalitis, intervertebral disc disease, spinal cord injury, bone marrow aplasia, non-regenerative anemia, atopic dermatitis, nephropathies and acute and chronic kidney disease, hepatitis, inflammatory bowel disease, endometritis, inflammatory heart disease, keratitis, gingivostomatitis, bronchitis, asthma, pneumonia, fibrosis, lupus, myopathies, tendinopathy, arthritis. Currently, the conventional treatment for most of those conditions is based on relieving the symptoms, however, an abundant number of scientific studies and clinical experience suggest that for most of chronic, inflammatory, degenerative diseases, conventional drugs do not provide complete pain relief and do not provide the cure. In contrast to drug therapy, cellular therapies such as GMSC therapy do not rely on a single target receptor or pathway for their action. Cellular therapy functions trophically by secreting cytokines and growth factors and by recruiting endogenous cells to the injured site, and it may promote cellular differentiation into the resident lineages. Mesenchymal stem cells, which include GMSC, are known to communicate with cells of their local environment, suppress immune reactions, and inhibit apoptosis. Recent studies also show that bone marrow stem cells (which have similar regenerative ability as GMSC) may be used to treat various veterinary species for) can deliver new mitochondria to damaged cells, thereby rescuing aerobic metabolism. Thus, GMSC therapy can reduce pain and enhance healing in a variety of diseases as cited but not limited to the ones above, resulting in improved quality of life. GMSC may be stored at the Veterinary clinic or animal hospitals for ease and speed of treatment for xenogeneic, allogeneic or autologous treatments.

Another use for GMSC is to suppress immune reactions in order to prevent graft-versus-host (GVH) disease. The GMSC can be used for regeneration of tissues or for the various factors and/or enzymes that it can secrete or for its effects on surrounding environment upon differentiation. GMSC or differentiated cells may be transplanted into the recipient where the cells will proliferate and differentiate to form new cells and tissues thereby providing the physiological processes normally provided by that tissue.

In another aspect of the present invention, the GMSC are administered with a pharmaceutically acceptable carrier or excipients. The pharmaceutically acceptable excipients described herein, for example, vehicles, adjuvants, carriers or diluents, are well-known to those who are skilled in the art and are readily available to the public. It is preferred that the pharmaceutically acceptable carrier or excipient be one which is chemically inert to the therapeutic composition and one which has no detrimental side effects or toxicity under the conditions of use. The choice of excipient or carrier will be determined in part by the particular therapeutic composition, as well as by the particular method used to administer the composition. Accordingly, there is a wide variety of suitable formulations of the pharmaceutical composition of the present invention. The formulations described herein are merely exemplary and are in no way limiting. Often the physiologically acceptable carrier is an aqueous pH buffered solution. Examples of physiologically acceptable carriers include saline, solvents, dispersion media, cell culture media, aqueous buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids Such as glycine, glutamine, asparagine, arginine or lysine; monosaccharides, disaccharides, and other carbohydrates including glucose, mannose, or dextrins; chelating agents such as EDTA: sugar alcohols such as mannitol or sorbitol; salt-forming counterions such as sodium; and/or nonionic surfactants such as TWEEN™, polyethylene glycol (PEG), and PLURON ICS™.

### Musculoskeletal Conditions

GMSC (genetically modified or not) and/or their derived vesicles, exosomes and conditioned media may also be administered to patients with or soft tissue injury including injury ligament injury, tendon injury. In one aspect of the invention, GMSC (genetically modified or not) and/or their derived vesicles, exosomes and conditioned media are injected at the desired location, e.g., in the surrounding area of the injured tissue or systemically which can be done but are not limited to subcutaneous, intramuscular injections and other medical devices, for example but not limited to catheter, with the help of ultrasound for precision or not. In another aspect of the invention the GMSC can be genetically modified in order to enhance the treatment outcome, for example but not limited to secret growth factors, to modulate the immune system, to reduce inflammation. In another aspect of the invention the GMSC genetically modified and/or their derived vesicles, exosomes and conditioned media can be also used to treat soft tissue injury. In another aspect of the invention the GMSC can be differentiated in order to enhance the treatment outcome, for example but not limited to osteogenic and chondrogenic lineages differentiation. In another aspect of the invention the use of support material such as matrigel, hydrogel, polymers and others, in an example but not limited to the hyaluronic acid can be introduced with GMSC and their differentiated cells, also in the genetically engineered GMSC. One of skill in the art (e.g., veterinarian) can adjust the volume of the suspension and the number of cells proportionally to the weight of the subject. An effective amount for systemic injections is in the range of 1-10 million cells per animal or 5 million/10 kg, depending on the size of the animal. As a non-limiting example of an effective amount for local injection is in the range of 1 million of GMSC per each square of injured tissue. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Muscular Dystrophy

GMSC (genetically modified or not) and/or their derived vesicles, exosomes and conditioned media may also be administered to patients with muscular dystrophy. In one aspect of the invention, GMSC (genetically modified or not) and/or their derived vesicles, exosomes and conditioned media are injected at the desired location, e.g., in the surrounding area of the injured tissue, with the help of ultrasound for precision or not, or systemically which can be done subcutaneous, intramuscular injections and other medical devices, for example but not limited to catheter. In another aspect of the invention the GMSC can be genetically modified in order to enhance the treatment outcome, for example but not limited to secret growth factors, to modulate the immune system, to reduce inflammation. In another aspect of the invention the GMSC genetically modified and/or their derived vesicles, exosomes and conditioned media can be also used to treat spinal cord injury. In another aspect of the invention the GMSC can be differentiated in order to enhance the treatment outcome, for example but not limited to muscle lineages differentiation. In another aspect of the invention the use of support material such as matrigel, hydrogel, polymers and others, in an example but not limited to the hyaluronic acid can be introduced with GMSC and their differentiated cells, also in the genetically engineered GMSC. One of skill in the art (e.g., veterinarian) can adjust the Volume of the Suspension and the number of cells proportionally to the weight of the subject. An effective amount for systemic injections is in the range of 1-10 million cells per animal or 5 million/10 kg, depending on the size of the animal. As a non-limiting example of an effective amount for local injection is in the range of 1 million of GMSC per each square of injured tissue. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Bone Marrow Disorders

GMSC (genetically modified or not) and/or their derived vesicles, exosomes and conditioned media may also be administered to patients with bone marrow disorders in a not limiting example aplasia, non-regenerative anemia, leukemia. In one aspect of the invention, GMSC (genetically modified or not) and/or their derived vesicles, exosomes and conditioned media are injected at the bone marrow or systemically which can be done but are not limited to subcutaneous, intramuscular injections and other medical devices, for example but not limited to catheter. In another aspect of the invention the GMSC can be genetically modified in order to enhance the treatment outcome, for example but not limited to secret growth factors, to modulate the immune system, to reduce inflammation. In another aspect of the invention the GMSC genetically modified and/or their derived vesicles, exosomes and conditioned media can be also used to treat bone marrow disorders. In another aspect of the invention the GMSC can be differentiated in order to enhance the treatment outcome, for example but not limited to blood lineages differentiation. In another aspect of the invention the use of support material such as matrigel, hydrogel, polymers and others, in an example but not limited to the hyaluronic acid can be introduced with GMSC and their differentiated cells, also in the genetically engineered GMSC. One of skill in the art (e.g., veterinarian) can adjust the Volume of the Suspension and the number of cells proportionally to the weight of the subject. An effective amount is in the range of 1-10 million cells per animal or 5 million/10 kg, depending on the size of the animal. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

As a non-limiting example, a 70-kg human patient can receive 5 million GMSC in 0.5 ml excipient directly in the bone marrow. In another aspect of the invention the use of support material such as matrigel, hydrogel, polymers and others. In another aspect of the invention the cells could be genetically modified to enhance clinical outcome for human patients as well.

### Osteoarthritis

GMSC (genetically modified or not) and/or their derived vesicles, exosomes and conditioned media may be used to treat patients for osteoarthritis (OA). OA is the most common cause of chronic pain in dogs, with more than 20%, or 10 to 12 million dogs, afflicted in the United States at any time. OA is characterized by degeneration of the articular cartilage, with loss of matrix, fibrillation, and formation of fissures, and can result in complete loss of the cartilage surface. While OA can afflict virtually any joints, it is most frequently associated with the stifle and hip joints. The treatment may be but is not limited to injection of GMSC (genetically modified or not), and/or their derived vesicles, exosomes and conditioned media into these joints, intramuscularly or subcutaneously in the surrounding injured area. In another aspect of the invention the GMSC can be genetically modified, in order to enhance the treatment outcome, for example but not limited to secret growth factors, to modulate the immune system, to reduce inflammation. In another aspect of the invention the GMSC genetically modified and/or their derived vesicles, exosomes and conditioned media can be also used to treat osteoarthritis. In another aspect of the invention the GMSC can be differentiated in order to enhance the treatment outcome in a non-limiting example of chondrogenic and osteogenic differentiation. It is well within the knowledge of one of skill in the art (e.g., veterinarian) to adjust the volume of the cell Suspension and the number of cells proportional to the size of the Subject. As a non-limiting example, a 20-kg dog can receive 4 million GMSC per joint. In another aspect of the invention the use of support material such as matrigel, hydrogel, polymers and others, in an example the hyaluronic acid can be introduced with GMSC into the site of treatment.

As a non-limiting example, a 70-kg human patient can receive 4 million GMSC in 0.5 ml excipient per joint. In another aspect of the invention the use of support material such as matrigel, hydrogel, polymers and others, in an example the hyaluronic acid can be introduced with GMSC into the site of treatment.

Following the treatment, owners are counseled to leash-walk their dogs 30 minutes daily. Any oral medications that the recipient is taking prior to GMSC treatment are allowed to continue. Any intra-articular treatments prior to GMSC treatment should discontinue. Post-treatment, veterinary evaluation will incorporate history, physical examination, and lameness examination including joint mobility and notation of pain on manipulation. Clinical outcome measures will be based on veterinary orthopedic evaluation using a numerical rating scale based on a standardized questionnaire. Baseline results for both owner and veterinary evaluations can be recorded between 2 and 14 days before the dogs receive either the test or control preparation by intra-articular injection. Follow-up visits to the veterinary clinic can be done at 30, 60, and 90 days after the dog's intra-articular injection. At each visit, owners can be asked to complete a numeric rating scale (1 (best) to 5 (worst)) as part of a standard questionnaire adapted from the Cincinnati Orthopedic Disability Index, which include evaluation of the following parameters: walk, run, jump, turning suddenly, getting up from lying down, lying down from standing, climbing stairs, descending stairs, squatting to urinate or defecate, stiffness in the morning, stiffness in the evening, difficulty walking on slippery floors, and willingness to play voluntarily.

### Heart Conditions

GMSC (genetically modified or not) and/or their derived vesicles, exosomes and conditioned media may also be administered to patients with heart conditions including but not limited to myocarditis, endocarditis, pericarditis and cardiomyopathies. In one aspect of the invention, GMSC (genetically modified or not) and/or their derived vesicles, exosomes and conditioned media are injected at the desired location, e.g., in the surrounding area of the injured heart or systemically which can be done but are not limited to subcutaneous, intramuscular injections, other medical devices, for example but not limited to catheter, with the help of ultrasound for precision or not. In another aspect of the invention the GMSC can be genetically modified in order to enhance the treatment outcome, for example but not limited to secret growth factors, to modulate the immune system. In another aspect of the invention the GMSC genetically modified and/or their derived vesicles, exosomes and conditioned media can be also used to treat cardiomyopathies. In another aspect of the invention the GMSC can be differentiated in order to enhance the treatment outcome, for example but not limited to heart lineages differentiation. In another aspect of the invention the use of support material such as matrigel, hydrogel, polymers and others, in an example but not limited to the hyaluronic acid can be introduced with GMSC and their differentiated cells, also in the genetically engineered GMSC. In another aspect of the invention the use of support material such as matrigel, hydrogel, polymers and others, in an example but not limited to the hyaluronic acid can be introduced with GMSC into the site of treatment. One of skill in the art (e.g., veterinarian) can adjust the Volume of the Suspension and the number of cells proportionally to the weight of the subject. An effective amount for systemic injections is in the range of 1-10 million cells per animal or 5 million/10 kg, depending on the size of the animal. As a non-limiting example of an effective amount for local injection is in the range of 1 million of GMSC per each square of injured tissue. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Neurological and Spinal Conditions

### Spinal Cord Injury

GMSC (genetically modified or not) and/or their derived vesicles, exosomes and conditioned media may also be administered to patients with spinal cord injury. In one aspect of the invention, GMSC (genetically modified or not) and/or their derived vesicles, exosomes and conditioned media are injected at the desired location, e.g., in the surrounding area of the injured tissue, with the help of ultrasound for precision or not, via epidural or systemically which can be done subcutaneous, intramuscular injections and other medical devices, for example but not limited to catheter. In another aspect of the invention the GMSC can be genetically modified in order to enhance the treatment outcome, for example but not limited to secret growth factors, to modulate the immune system, to reduce inflammation. In another aspect of the invention the GMSC genetically modified and/or their derived vesicles, exosomes and conditioned media can be also used to treat spinal cord injury. In another aspect of the invention the GMSC can be differentiated in order to enhance the treatment outcome, for example but not limited to neurogenic lineages differentiation. In another aspect of the invention the use of support material such as matrigel, hydrogel, polymers and others, in an example the hyaluronic acid can be introduced with GMSC and their differentiated cells, also in the genetically engineered GMSC. One of skill in the art (e.g., veterinarian) can adjust the Volume of the Suspension and the number of cells proportionally to the weight of the subject. An effective amount for systemic injections is in the range of 1-10 million cells per animal or 5 million/10 kg, depending on the size of the animal. As a non-limiting example of an effective amount for local injection is in the range of 1 million of cells per each square of injured tissue. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Other Neurological Conditions

GMSC (genetically modified or not) and/or their derived vesicles, exosomes and conditioned media may also be administered to patients with neurological conditions including but not limited to distemper virus, cognitive dysfunction, dementia, alzheimer, granulomatous meningoencephalitis. In one aspect of the invention, non-limited examples of GMSC (genetically modified or not) and/or their derived vesicles, exosomes and conditioned media injection are local via epidural, intra thecal, subcutaneous, intramuscular injections and other medical devices, for example but not limited to catheter. In another aspect of the invention the GMSC can be genetically modified in order to enhance the treatment outcome, for example but not limited to secret growth factors, to modulate the immune system, to reduce inflammation. In another aspect of the invention the GMSC genetically modified and/or their derived vesicles, exosomes and conditioned media can be also used to treat neurological conditions. In another aspect of the invention the GMSC can be differentiated in order to enhance the treatment outcome, for example but not limited to neurologic lineages differentiation. In another aspect of the invention the use of support material such as matrigel, hydrogel, polymers and others, in an example but not limited to the hyaluronic acid can be introduced with GMSC and their differentiated cells, also in the genetically engineered GMSC. One of skill in the art (e.g., veterinarian) can adjust the Volume of the Suspension and the number of cells proportionally to the weight of the subject. An effective amount for systemic injections is in the range of 1-10 million cells per animal or 5 million/10 kg, depending on the size of the animal. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Intervertebral Disc Disease

GMSC genetically modified or not) and/or their derived vesicles, exosomes and conditioned media may also be administered to patients with intervertebral disc disease. In one aspect of the invention, GMSC (genetically modified or not) are injected at the desired location, e.g., in the surrounding area of the injured tissue, with the help of ultrasound for precision or not, or systemically which can be done subcutaneous, intramuscular injections and other medical devices, for example but not limited to catheter. In another aspect of the invention the GMSC can be genetically modified in order to enhance the treatment outcome, for example but not limited to secret growth factors, to modulate the immune system, to reduce inflammation. In another aspect of the invention the GMSC genetically modified and/or their derived vesicles, exosomes and conditioned media can be also used to treat intervertebral disc disease. In another aspect of the invention the GMSC can be differentiated in order to enhance the treatment outcome, for example but not limited to chondrogenic lineages differentiation. In another aspect of the invention the use of support material such as matrigel, hydrogel, polymers and others, in an example but not limited to the hyaluronic acid can be introduced with GMSC and their differentiated cells, also in the genetically engineered GMSC. One of skill in the art (e.g., veterinarian) can adjust the Volume of the Suspension and the number of cells proportionally to the weight of the subject. An effective amount for systemic injections is in the range of 1-10 million cells per animal or 5 million/10 kg, depending on the size of the animal. As a non-limiting example of an effective amount for local injection is in the range of 1 million of GMSC per each square of injured tissue. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Diseases of Internal Organs

### Lung Diseases

GMSC (genetically modified or not) and/or their derived vesicles, exosomes and conditioned media may also be administered to patients with lung diseases as a non-limiting example bronchitis, asthma, pneumonia, fibrosis. GMSC (genetically modified or not) and/or their derived vesicles, exosomes and conditioned media are injected for example but are not limited to via subcutaneous, intramuscular injections or intranasal, endotracheal. In another aspect of the invention the GMSC can be genetically modified in order to enhance the treatment outcome, for example but not limited to secrete growth factors, to reduce allergy and to modulate the immune systems. In another aspect of the invention the GMSC genetically modified and/or their derived vesicles, exosomes and conditioned media can be also used to treat lung diseases. In another aspect of the invention the GMSC can be differentiated in order to enhance the treatment outcome, for example epithelial differentiation. In another aspect of the invention the use of support material such as matrigel, hydrogel, polymers and others, in an example but not limited to the hyaluronic acid can be introduced with GMSC and their differentiated cells, also in the genetically engineered. One of skill in the art (e.g., veterinarian) can adjust the Volume of the Suspension and the number of cells proportionally to the weight of the subject. An effective amount is in the range of 1-10 million cells per animal or 5-20 million/10 kg, depending on the size of the animal and the injury intensity. After the treatment procedure, the owner of the subject should take care to monitor the subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Nephropathies

GMSC (genetically modified or not) and/or their derived vesicles, exosomes and conditioned media may also be administered to patients with nephropathies in a non-limited example are acute and chronic kidney diseases. In one aspect of the invention, GMSC (genetically modified or not) and/or their derived vesicles, exosomes and conditioned media are injected at the desired location, e.g., in the surrounding area of the injured kidney, with the help of ultrasound for precision or not, or systemically which can be done subcutaneous, intramuscular injections, other medical devices, for example but not limited to catheter. In another aspect of the invention the GMSC can be genetically modified in order to enhance the treatment outcome, for example but not limited to secret growth factors, to modulate the immune system. In another aspect of the invention the GMSC genetically modified and/or their derived vesicles, exosomes and conditioned media can be also used to treat nephropathies. In another aspect of the invention the GMSC can be differentiated in order to enhance the treatment outcome, for example but not limited to kidney lineage differentiation. In another aspect of the invention the use of support material such as matrigel, hydrogel, polymers and others, in an example but not limited to the hyaluronic acid can be introduced with GMSC and their differentiated cells, also in the genetically engineered GMSC. In another aspect of the invention the use of support material such as matrigel, hydrogel, polymers and others, in an example the hyaluronic acid can be introduced with GMSC into the site of treatment. One of skill in the art (e.g., veterinarian) can adjust the Volume of the Suspension and the number of cells proportionally to the weight of the subject. An effective amount for systemic injections is in the range of 5-10 million cells per animal or 10 million/10 kg, depending on the size of the animal. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Liver Diseases

GMSC (genetically modified or not) and/or their derived vesicles, exosomes and conditioned media may also be administered to patients with liver diseases. In one aspect of the invention, GMSC (genetically modified or not) and/or their derived vesicles, exosomes and conditioned media are injected at the desired location, e.g., in the surrounding area of the injured liver, with the help of ultrasound for precision or not, or systemically which can be done but are not limited to subcutaneous, intramuscular injections, other medical devices, for example but not limited to catheter. In another aspect of the invention the GMSC can be genetically modified in order to enhance the treatment outcome, for example but not limited to secret growth factors, to modulate the immune system. In another aspect of the invention the GMSC genetically modified and/or their derived vesicles, exosomes and conditioned media can be also used to treat liver diseases. In another aspect of the invention the GMSC can be differentiated in order to enhance the treatment outcome, for example but not limited to liver lineage differentiation. In another aspect of the invention the use of support material such as matrigel, hydrogel, polymers and others, in an example but not limited to the hyaluronic acid can be introduced with GMSC and their differentiated cells, also in the genetically engineered GMSC. In another aspect of the invention the use of support material such as matrigel, hydrogel, polymers and others, in an example the hyaluronic acid can be introduced with GMSC into the site of treatment. One of skill in the art (e.g., veterinarian) can adjust the Volume of the Suspension and the number of cells proportionally to the weight of the subject. An effective amount for systemic injections is in the range of 5-10 million cells per animal or 10 million/10 kg, depending on the size of the animal. As a non-limiting example of an effective amount for local injection is in the range of 1 million of GMSC per each square of injured tissue. After the treatment procedure, the owner of the subject should take care to monitor the Subject on a daily basis to observe the following characteristics: monitoring the site of injection for signs of reaction Such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Endometritis and Endometriosis

GMSC (genetically modified or not) and/or their derived vesicles, exosomes and conditioned media may also be administered to patients with endometritis and endometriosis. In one aspect of the invention, GMSC and/or their derived vesicles, exosomes and conditioned media are (genetically modified or not) injected at the desired location, e.g., in the surrounding area of the injured tissue, with the help of ultrasound for precision or not, systemically which can be done subcutaneous, intramuscular injections, other medical devices, for example catheter or by uterus flushing. In another aspect of the invention the GMSC can be genetically modified in order to enhance the treatment outcome, for example but not limited to secret growth factors, to modulate the immune system. In another aspect of the invention the GMSC genetically modified and/or their derived vesicles, exosomes and conditioned media can be also used to treat endometritis and endometriosis. In another aspect of the invention the GMSC can be differentiated in order to enhance the treatment outcome. In another aspect of the invention the use of support material such as matrigel, hydrogel, polymers and others, in an example but not limited to the hyaluronic acid can be introduced with GMSC and their differentiated cells, also in the genetically engineered GMSC. In another aspect of the invention the use of support material such as matrigel, hydrogel, polymers and others, in an embodiment the hyaluronic acid can be introduced with GMSC into the site of treatment. One of skill in the art (e.g., veterinarian) can adjust the Volume of the Suspension and the number of cells proportionally to the weight of the subject. An effective amount for systemic injections is in the range of 5-10 million cells per animal or 10 million/10 kg, depending on the size of the animal. After the treatment procedure, the owner of the subject should take care to monitor the subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Idiopathic and Autoimmune Conditions

### Inflammatory Bowel Disease

GMSC (genetically modified or not) and/or their derived vesicles, exosomes and conditioned media may also be administered to patients with inflammatory bowel disease. In one aspect of the invention, GMSC (genetically modified or not) and/or their derived vesicles, exosomes and conditioned media are injected at the desired location, e.g., in the surrounding area of the injured tissue, with the help of ultrasound for precision or not, or systemically which can be done subcutaneous, intramuscular injections, other medical devices, for example but not limited to catheter. In another aspect of the invention the GMSC can be genetically modified in order to enhance the treatment outcome, for example secret growth factors, to modulate the immune system. In another aspect of the invention the GMSC genetically modified and/or their derived vesicles, exosomes and conditioned media can be also used to treat inflammatory bowel disease. In another aspect of the invention the GMSC can be differentiated in order to enhance the treatment outcome, for example bowel, epithelial, lineages differentiation. In another aspect of the invention the use of support material such as matrigel, hydrogel, polymers and others, in an example but not limited to the hyaluronic acid can be introduced with GMSC and their differentiated cells, also in the genetically engineered GMSC. In another aspect of the invention the use of support material such as matrigel, hydrogel, polymers and others, the hyaluronic acid can be introduced with GMSC into the site of treatment. One of skill in the art (e.g., veterinarian) can adjust the Volume of the Suspension and the number of cells proportionally to the weight of the subject. An effective amount for systemic injections is in the range of 5-10 million cells per animal or 10 million/10 kg, depending on the size of the animal. As a non-limiting example of an effective amount for local injection is in the range of 1 million of GMSC per each square of injured tissue. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Atopic Dermatitis

GMSC (genetically modified or not) and/or their derived vesicles, exosomes and conditioned media may also be administered to patients with atopic dermatitis. GMSC (genetically modified or not) and/or their derived vesicles, exosomes and conditioned media are injected systemically for example to subcutaneous, intramuscular injections. In another aspect of the invention the GMSC can be genetically modified in order to enhance the treatment outcome, for example secrete growth factors, to reduce allergy and to modulate the immune systems. In another aspect of the invention the GMSC genetically modified and/or their derived vesicles, exosomes and conditioned media can be also used to treat atopic dermatitis. In another aspect of the invention the GMSC can be differentiated in order to enhance the treatment outcome, for example immune lineage differentiation. In another aspect of the invention the use of support material such as matrigel, hydrogel, polymers and others, in an example but not limited to the hyaluronic acid can be introduced with GMSC and their differentiated cells, also in the genetically engineered. One of skill in the art (e.g., veterinarian) can adjust the Volume of the Suspension and the number of cells proportionally to the weight of the subject. An effective amount is in the range of 1-10 million cells per animal or 5 million/10 kg, depending on the size of the animal. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Diabetes

GMSC (genetically modified or not) and/or their derived vesicles, exosomes and conditioned media may also be administered to veterinary species and even humans with diabetes. It is general information about the benefits of mesenchymal stem cells for type 1 diabetic patients. In one aspect of the invention, GMSC treatment can prevent the progression in newly diagnosed patients as well as immunomodulation in any patients. In one aspect of the invention, GMSC (genetically modified or not) and/or their derived vesicles, exosomes and conditioned media are injected at the desired location, e.g., in the surrounding area of the pancreas, which can be done with the help of ultrasound for precision or not and can be done systemically for example subcutaneous, intramuscular injections. In another aspect of the invention the GMSC can be genetically modified in order to enhance the treatment outcome, for example but not limited to secret insulin and to modulate the immune systems. In another aspect of the invention the GMSC genetically modified and/or their derived vesicles, exosomes and conditioned media can be also used to treat diabetes. In another aspect of the invention the GMSC can be differentiated in order to enhance the treatment outcome, for example pancreatic lineage differentiation. In another aspect of the invention the use of support material such as matrigel, hydrogel, polymers and others, in an example but not limited to the hyaluronic acid can be introduced with GMSC and their differentiated cells, also in the genetically engineered One of skill in the art (e.g., veterinarian) can adjust the Volume of the Suspension and the number of cells proportionally to the weight of the subject. An effective amount is in the range of 1-10 million cells per animal or 5 million/10 kg, depending on the size of the animal. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30, 55-60, 90, 120 days after treatment.

### Lupus

GMSC (genetically modified or not) and/or their derived vesicles, exosomes and conditioned media may also be administered patients with lupus. GMSC and/or their derived vesicles, exosomes and conditioned media are injected for example but are not limited to via subcutaneous, intramuscular injections. In another aspect of the invention the GMSC can be genetically modified in order to enhance the treatment outcome, for example to secrete growth factors, to reduce allergy and to modulate the immune systems. In another aspect of the invention the GMSC genetically modified and/or their derived vesicles, exosomes and conditioned media can be also used to treat lupus. In another aspect of the invention the GMSC can be differentiated in order to enhance the treatment outcome. In another aspect of the invention the use of support material such as matrigel, hydrogel, polymers and others, in an example the hyaluronic acid can be introduced with GMSC and their differentiated cells, also in the genetically engineered. One of skill in the art (e.g., veterinarian) can adjust the Volume of the Suspension and the number of cells proportionally to the weight of the subject. An effective amount is in the range of 1-10 million cells per animal or 5-20 million/10 kg, depending on the size of the animal and the condition intensity. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Gingivostomatitis

GMSC (genetically modified or not) and/or their derived vesicles, exosomes and conditioned media may also be administered to patients with gingivostomatitis. In another aspect of the invention the GMSC can be genetically modified in order to enhance the treatment outcome, for example but not limited to secret growth factors, to modulate the immune system, to reduce inflammation. In another aspect of the invention the GMSC genetically modified and/or their derived vesicles, exosomes and conditioned media can be also used to treat gingivostomatitis. In another aspect of the invention the GMSC can be differentiated in order to enhance the treatment outcome. In one aspect of the invention, GMSC (genetically modified or not) and/or their derived vesicles, exosomes and conditioned media are injected at the desired location, e.g., in the gingiva or systemically which can be done subcutaneously, intramuscularly. In another aspect of the invention the use of support material such as matrigel, hydrogel, polymers and others, in an example the hyaluronic acid can be introduced with GMSC into the site of treatment. It is well within the knowledge of one of skill in the art (e.g., veterinarian) to adjust the volume of the cell suspension and the number of cells proportional to the size of the Subject and the injury itself. As a non-limiting example of an effective amount is in the range of 1-10 million of GMSC per each square of injured tissue, depending on the size of the patient and the intensity of the condition or 1-10 million/patient. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Ophthalmic Conditions

GMSC (genetically modified or not) and/or their derived vesicles, exosomes and conditioned media may also be administered to veterinary species and even humans with eye disorders, including keratitis sicca, keratitis eosinophilic, corneal ulcer, macular degeneration. In another aspect of the invention the GMSC can be genetically modified in order to enhance the treatment outcome, for example but not limited to secret growth factors, to modulate the immune system, to reduce inflammation. In another aspect of the invention the GMSC genetically modified and/or their derived vesicles, exosomes and conditioned media can be also used to treat eye disorders. In another aspect of the invention the GMSC can be differentiated in order to enhance the treatment outcome in a non-limiting example in retinal lineage differentiation. In one aspect of the invention, GMSC (genetically modified or not) and/or their derived vesicles, exosomes and conditioned media are injected at the desired location, e.g., in the retina, eye bulb, topically (in a non-limited example in an eye drop solution), subconjunctivally, intraviterally, or systemically which can be done subcutaneously, intramuscularly. In another aspect of the invention the use of support material such as matrigel, hydrogel, polymers and others, in an example the hyaluronic acid can be introduced with GMSC into the site of treatment. It is well within the knowledge of one of skill in the art (e.g., veterinarian) to adjust the volume of the cell suspension and the number of cells proportional to the size of the Subject and the injury itself. As a non-limiting example of an effective amount is in the range of 1-10 million of GMSC per each square of injured tissue, depending on the size of the patient and the intensity of the condition. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 days after treatment.

### Other Conditions

### Urinary Incontinence

GMSC (genetically modified or not) and/or their derived vesicles, exosomes and conditioned media may be used to treat patients with urinary incontinence. Stem cell therapy for UI has been successfully demonstrated in human medicine. Accordingly, GMSC and/or their derived vesicles, exosomes and conditioned media can be used to treat UI. In another aspect of the invention the GMSC can be genetically modified in order to enhance the treatment outcome, for example to secrete growth factors, to modulate the immune system, to reduce inflammation. In another aspect of the invention the GMSC genetically modified and/or their derived vesicles, exosomes and conditioned media can be also used to treat UI. In another aspect of the invention the GMSC can be differentiated in order to enhance the treatment outcome in a non-limiting example of muscle and neurogenic lineages differentiation. The subject being treated for UI has an effective amount of GMSC (genetically modified or not) injected into but not limited to the sphincter area of the urethra. The GMSC (genetically modified or not) can be injected subcutaneously, intramuscularly in the surroundings of the sphincter area of the urethra. Precision injection can be accomplished by using an uroscope that enables both visualization and injection. One of skill in the art (e.g., veterinarian) can adjust the Volume of the Suspension and the number of cells proportionally to the weight of the subject. An effective amount is in the range of 1-10 million cells per animal or 5 million/10 kg, depending on the size of the animal. In another aspect of the invention the use of support material such as matrigel, hydrogel, polymers and others, in an example the hyaluronic acid can be introduced with GMSC into the site of treatment. After the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction and urinary blockage, any other unusual signs in the animal such as persistent nausea, fatigue, lethargy, decreased appetite and difficult urination. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 10-15, 25-30 and 55-60 days after treatment.

### Healing Wounds

GMSC (genetically modified or not) and/or their derived vesicles, exosomes and conditioned media may also be administered to patients for the purpose of accelerating wound healing and reducing scar formation. This has application for improved cosmesis (i.e. to improve scar healing purely for cosmetic purposes), as an adjunct to improve surgical scars within the body (e.g. excessive scarring at surgical anastomosis sites can lead to surgical complications, such as anastomosis site contractures, for naturally occurring wounds in any tissue, non-limiting examples of acute wounds that can be treated with the present invention include wounds in the corneal tissue, month mucosa, stomach mucosa, colon mucosa, skin, abrasions, avulsions, contusions, crush wounds, cuts, lacerations, projectile wounds and puncture wounds. Chronic wounds include, but are not limited to, diabetic skin sores, pressure sores, surgical wounds, spinal injury wounds, burns, chemical-induced wounds and wounds due to blood vessel disorders. Local injection of GMSC and/or their derived vesicles, exosomes and conditioned media at the time of surgery, or, in a delayed fashion, can improve surgical tissue site healing and reduce the incidence of post-Surgical complications due to excessive scarring). In another aspect of the invention the GMSC can be genetically modified in order to enhance the treatment outcome, for example but not limited to secret growth factors, to modulate the immune system, to reduce inflammation. In another aspect of the invention the GMSC genetically modified and/or their derived vesicles, exosomes and conditioned media can be also used to treat wounds. In another aspect of the invention the GMSC can be differentiated in order to enhance the treatment outcome. In one aspect of the invention, GMSC (genetically modified or not) and/or their derived vesicles, exosomes and conditioned media are injected at the desired location, e.g., in the surrounding area of the wound, which can be done but are not limited to subcutaneously, intramuscularly, subconjunctivally injections. In another aspect of the invention the use of support material such as matrigel, hydrogel, polymers and others, in an example but not limited to the hyaluronic acid can be introduced with GMSC into the site of treatment. It is well within the knowledge of one of skill in the art (e.g., veterinarian) to adjust the volume of the cell suspension and the number of cells proportional to the size of the Subject and the injury itself. As a non-limiting example of an effective amount is in the range of 1 million of GMSC per approximately each square of injured tissue, after the treatment procedure, the owner of the subject should take care to monitor the Subject on a daily basis to observe the following characteristics: monitoring the site of injection for signs of reaction such as redness, pain, and heat, monitoring the animal for signs of anaphylactic reaction or any other unusual signs in the animal. If any of these characteristics are observed, then the owner of the subject should inform the veterinarian immediately so that the veterinarian can take the appropriate actions to bring the subject into a better state of health. Veterinarians' evaluation can be conducted at regular intervals after the treatment, for example, at 24 h, 48 h, 72 h, 5 days, 10-15, 25-30 and 55-60 days after treatment.

### Prophylactic Uses for GMSC

Prophylactic Uses for GMSC (genetically modified or not) and/or their derived vesicles, exosomes and conditioned media may also used as a prophylaxis to delay the development and progression of chronic and degenerative diseases, including but not limited to hip dysplasia and avascular necrosis of femoral head, kidney diseases, neurological conditions, degenerative myopathies by administering an effective amount of GMSC closest to injured are as possible. An effective amount is in the range of 1-10 million cells per animal or 5 million/10 kg, depending on the size of the animal, which is readily determined by one of skill in the art (e.g., a veterinarian).

### EXAMPLES

### Example 1. Treatment of Tendinopathy

An 8 year old male horse suffers from tendinopathy, in his left fore leg tenderness on palpation and pain, often when exercising or with movement. The horse exhibits a limp. To treat the animal, allogeneic GMSC at a dose of 3 million cells are administered via injection to the affected tendon. Within a week, the horse is no longer exhibiting signs of pain or tenderness in his left foreleg.

### Example 2. Treatment of Tendinopathy

A 4 year old female dog suffers from tendinopathy in her right fore leg tenderness on palpation and pain, often when exercising or with movement. The dog exhibits a limp. To treat the animal, allogeneic GMSC at a dose of one million cells are administered via injection to the affected tendon. Within a week, the dog is no longer exhibiting signs of pain or tenderness in her right foreleg.

### Example 3. Treatment of Rheumatoid Arthritis

A 14 year old dog suffers from arthritis. Autologous GMSC are administered at a dose of one million cells via intra-articular. Within a week, the patient's arthritis symptoms decrease.

### Example 4. Treatment of Lupus

A 3 year old cat suffers from lupus. Allogenic GMSC at a dose of one million cells/kg are administered via injection. Within a week, the patient's lupus symptoms decrease.

### Example 5. Treatment of Tendinopathy

A 1 year old female horse suffers from a ligament tear in his left rear leg tenderness on palpation and pain, often when exercising or with movement. The horse exhibits a limp. To treat the animal, xenogenic GMSC at a dose of 10 million cells are administered via injection to the affected area. Within two weeks, the horse is no longer exhibiting signs of pain or tenderness in her left leg.

### Example 6. Treatment of Recurrent Uveitis

A 2 year old female horse suffers from Equine Recurrent Uveitis (ERU). To treat the animal, xenogenic GMSC at a dose of 5 million cells are administered via injection to the affected area. Within a week, the patient's ERU symptoms decrease.

### Example 7. Energy Boost

A 12 year old male horse is lethargic. To treat the animal, allogeneic GMSC at a dose of 50 million cells are administered via injection. Within a week, the patient is more energetic.

### Example 8. Treatment of Atopic Dermatitis

A 4 year old dog suffers from atopic dermatitis. Autologous GMSC are administered at a dose of one million cells/kg via injection. Within a week, the patient's symptoms decrease.

### Example 9. Treatment of Atopic Dermatitis

A 13 year old dog suffers from atopic dermatitis. Allogenic GMSC are administered at a dose of one million cells/kg via injection. Within two weeks, the patient's symptoms decrease.

### Example 10. Treatment of Atopic Dermatitis

A 9 year old dog suffers from atopic dermatitis. Xenogenic GMSC are administered at a dose of one million cells/kg via injection. Within two weeks, the patient's symptoms decrease.

### Example 11. Treatment of Atopic Dermatitis

A 9 year old dog suffers from atopic dermatitis. Genetically modified Xenogenic GMSC are administered at a dose of one million cells/kg via injection. Within one week, the patient's symptoms decrease.

### Example 12. Treatment of Immune Mediated Nephropathy

A 5 year old cat suffers from immune mediated nephropathy. Allogenic GMSCs are administered at a dose of one million cells/kg via injection. Within three weeks, the patient's symptoms decrease.

### Example 13. Treatment of Gingivostomatitis

A 9 year old cat suffers from gingivostomatitis. Xenogenic GMSC are administered at a dose of 5 million via injection. Within one week, the patient's symptoms decrease.

### Example 14. Treatment of Chronic Kidney Disease

A 7 year old cat suffers from chronic kidney disease. Allogeneic MSCs are administered at a dose of one million cells/kg via injection. Within two weeks, the patient's symptoms decrease.

### Example 15. The Use of GMSC Genetically Modified to Produce IL-10

GMSC are harvested as described supra, then they are genetically induced to produce IL-10, in order to create a cell product with maximum anti-inflammatory properties.

### Example 16. Treatment of Atopic Dermatitis with Genetically Modified GMSC

A 9 year old dog suffers from atopic dermatitis. Xenogenic genetically modified GMSC are administered at a dose of one million cells/kg via injection. Within less than a week, the patient's symptoms decrease

### Example 17. The Use of IL-10-Producing GMSC

GMSC are harvested as described supra, MSC are then isolated and can be cultured with molecules to stimulate their ability to produce IL-10, in order to create a cell product with maximum anti-inflammatory properties.

### Example 18. Isolation of VEGF-Producing MSC

GMSC are harvested as described supra. MSC are then isolated and can be cultured with molecules to stimulate their ability to produce VEGF to create a cell product with maximum angiogenic property. The product is used for diabetic wounds and ischemic diseases.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A method of isolating animal gonadal mesenchymal stem cells comprising:
a) isolating animal gonadal tissue;
b) selecting mesenchymal stem cells in the animal gonadal tissue based on their rapid adherence to culture plastic; and
c) isolating the selected mesenchymal stem cells.

2. The method of claim 1 which further comprises culturing the isolated mesenchymal stem cells for a first select period of time in a culture media comprising serum followed by a second period of time in a serum free culture wherein media and vesicles are collected from the second period of time culture and added to a pharmaceutical composition.

3. The method of claim 2 wherein the culture media contains at least one stimulating agent.

4. The method of claim 3 wherein the stimulating agent is selected from the group consisting of cytokines and reactive proteins.

5. The method of claim 1 wherein the isolated mesenchymal stem cells are used to treat a physical condition in an animal or in a human.

6. The method of claim 1 wherein the cells are autologous to the treated animal.

7. The method of claim 1 wherein the cells are allogeneic to the treated animal or human.

8. The method of claim 1 wherein the cells are xenogeneic to the treated animal or human.

9. The method of claim 1 wherein the physical condition selected from the group comprising:
a) a dermatological condition;
b) a musculoskeletal condition;
c) an ophthalmic condition;
d) a renal condition;
e) an endocrine condition;
f) a hepatic condition;
g) a neurological condition;
h) a gastro intestinal condition;
i) an immunological/autoimmune condition;
j) an urogenital condition; and
k) a respiratory condition.

10. A composition for treating a physical condition in an animal or human comprising animal gonadal mesenchymal stem cells isolated from animal gonadal tissue.

11. The composition according to claim 8 wherein the mesenchymal stem cells are cultured for a first select period of time in a culture media comprising serum followed by a second period of time in a serum free culture wherein media and vesicles are collected from the second period of time culture and added to the composition or used in a second composition.

12. A composition according to claim 8 wherein the isolated mesenchymal stem cells are genetically modified.
